(19) United States
(12) Patent Application Publication
(10) Pub. No.: US 2025/0369039 A1
(43) Pub. Date: Dec. 4, 2025

(54) METHODS AND SYSTEMS FOR DETECTING RIBONUCLEIC ACIDS

(71) Applicant: UNM RAINFOREST INNOVATIONS, Albuquerque, NM (US)

(72) Inventor: Christos ARGYROPOULOS, Albuquerque, NM (US)

(73) Assignee: UNM RAINFOREST INNOVATIONS, Albuquerque, NM (US)

(21) Appl. No.: 18/851,909

(22) PCT Filed: Mar. 31, 2023
(86) PCT No.: PCT/US2023/017049

§ 371 Date: Sep. 27, 2024

Related U.S. Application Data
(60) Provisional application No. 63/326,157, filed on Mar. 31, 2022.

(51) Int. Cl. C12Q 1/6869

(52) U.S. Cl. CPC C12Q 1/6869

(57) ABSTRACT

Provided herein are methods of simultaneously detecting coding and non-coding ribonucleic acids (RNAs) in a sample that include attaching a polymeric nucleic acid tail to a plurality of the non-coding linear RNAs in the sample to produce a population of RNA molecules that each comprise polymeric nucleic acid tails. The methods also include obtaining sequence information from the population of RNA molecules that each comprise polymeric nucleic acid tails and/or from derivative nucleic acid molecules thereof irrespective of lengths of the RNA molecules or the derivative nucleic acid molecules thereof using a long-read sequencing technique. Related methods, systems, and computer readable media are also provided.

### CROSS-REFERENCE TO RELATED APPLICATIONS

This application is a U.S. National Phase application of PCT/US2023/017049, filed Mar. 31, 2023, which claims the benefit of U.S. Provisional Patent Application No. 63/326,157, filed on Mar. 31, 2022, the disclosures of which are incorporated by reference herein in their entireties.

### BACKGROUND

Long coding and noncoding (short or long >200 nt long) RNAs yield valuable information about the abundance and novelty of the transcriptome and its epigenetic regulation respectively. Noncoding RNAs are of interest for clinical research applications, as their relative stability and tissue-specific nature make them viable candidates for disease-state biomarkers. There is currently a need to simultaneously sequence coding and non-coding RNAs from the same sample in a convenient and robust manner. In particular, consideration of epigenetic regulation often requires examination of the quantitative relationships between noncoding and coding RNAs or between categories of noncoding RNAs, e.g., microRNAs and long noncoding (lncRNA) RNAs. On the biomarker side, there exist multiple proposals for cell-free RNA based panels derived from either microRNAs or lncRNAs, but there has been little work to combine markers from both categories, or even coding RNAs and evaluate them in a prospective rigorous manner. This is in no small part due to the biochemical incompatibility of the existing sequencing protocols for non-coding and coding RNAs, which generally require construction of separate libraries that are then sequenced in parallel.

Approaches to simultaneously sequence RNAs from multiple classes like Holo-Seq and Smart-seq-total target short-read sequencing platforms. In recent years, long-read platforms such as those by Oxford Nanopore Technologies (ONT) that span the entire range from portable devices to large scale high throughput sequencers have emerged as an alternative to short read sequencing. While the spectrum of applications of Nanopore sequencing is extremely wide, ranging from genomic sequencing to epigenomics and transcriptomics, there currently does not exist a method to simultaneously profile short and long RNAs in this platform. In fact, most library protocols for Nanopore sequencing exclude cDNAs derived from short RNAs. This represents a significant missed opportunity, because Nanopore sequencing provides the most accessible platform, in terms of acquisition, maintenance and operational costs, with a portability profile that is unmatched by all other alternatives.

Accordingly, it is apparent that there is a need for simultaneous sequencing of short and long RNAs.

### SUMMARY

The present disclosure provides methods, computer readable media, and systems that are useful in simultaneously sequencing both short and long ribonucleic acids (RNAs) in the same experimental run (e.g., in the same reaction mixture or container), unlike other approaches, which involve separate sequencing experiments given the different physical characteristics of RNA species from biological or other sample types. Some embodiments provide library preparation methods capable of simultaneously profiling short and long RNA reads in the same library on the nanopore sequencing platforms and provide related bioinformatics workflows to support the goals of RNA quantification. These and other attributes will be apparent upon complete review of the present disclosure, including the accompanying figures.

In one aspect, this disclosure provides a method of substantially simultaneously detecting coding and non-coding linear ribonucleic acids (RNAs) in a sample. The method includes attaching a polymeric nucleic acid tail to a plurality of the non-coding linear RNAs in the sample, wherein the sample comprises the coding and non-coding linear RNAs irrespective of lengths of the RNAs, to produce a population of RNA molecules that each comprise polymeric nucleic acid tails, and obtaining sequence information from the population of RNA molecules that each comprise polymeric nucleic acid tails and/or from derivative nucleic acid molecules thereof irrespective of lengths of the RNA molecules or the derivative nucleic acid molecules thereof using a long read sequencing technique, thereby substantially simultaneously detecting the coding and non-coding linear RNAs in the sample.

In one aspect, this disclosure provides a method of processing sequencing reads. The method includes attaching a polymeric nucleic acid tail to a plurality of the non-coding RNAs in a sample, wherein the sample comprises coding and non-coding ribonucleic acids (RNAs), to produce a population of RNA molecules that each comprise polymeric nucleic acid tails, obtaining sequencing reads from the population of RNA molecules that each comprise polymeric nucleic acid tails and/or from derivative nucleic acid molecules thereof, differentiating decorator sequence information from insert sequence information in the plurality of sequencing reads, and determining orientations of subsequences in the plurality of sequencing reads corresponding to the coding and non-coding RNAs using the decorator and/or insert sequence information, thereby the processing sequencing reads.

In one aspect, this disclosure provides a method of mapping sequence information to a genomic transcriptome using a computer. The method includes receiving, by the computer, sequencing reads from a population of ribonucleic acid (RNA) molecules that each comprise polymeric nucleic acid tails and/or from derivative nucleic acid molecules thereof, wherein the RNA molecules comprise coding and non-coding RNAs, differentiating, by the computer, decorator sequence information from insert sequence information in the plurality of sequencing reads, determining, by the computer, orientations of coding RNA sequence information and non-coding RNA sequence information from the insert sequence information, removing or disregarding, by the computer, decorator sequence information from the insert sequence information to produce processed insert sequence information, and mapping, by the computer, the processed insert sequence information to a selected genomic transcriptome, thereby mapping the sequence information to the genomic transcriptome.

In one aspect, this disclosure provides a method of detecting non-coding linear ribonucleic acids (RNAs) in a sample. The method includes attaching a polymeric nucleic acid tail to a plurality of the non-coding linear RNAs in the sample, wherein the sample comprises the coding and non-coding linear RNAs irrespective of lengths of the RNAs, to produce a population of RNA molecules that each comprise polymeric nucleic acid tails, and obtaining sequence information from the population of RNA molecules that each comprise polymeric nucleic acid tails and/or from derivative nucleic acid molecules thereof irrespective of lengths of the RNA molecules or the derivative nucleic acid molecules thereof using a sequencing technique, thereby detecting the non-coding linear RNAs in the sample.

In one aspect, this disclosure provides a system, comprising at least one controller that comprises, or is capable of accessing, computer readable media comprising non-transitory computer-executable instructions which, when executed by at least one electronic processor, perform at least: receiving sequencing reads from a population of ribonucleic acid (RNA) molecules that each comprise polymeric nucleic acid tails and/or from derivative nucleic acid molecules thereof, wherein the RNA molecules comprise coding and non-coding RNAs, differentiating decorator sequence information from insert sequence information in the plurality of sequencing reads, and determining orientations of subsequences in the plurality of sequencing reads corresponding to the coding and non-coding RNAs using at least the insert sequence information.

In another aspect, the disclosure provides a system, comprising at least one controller that comprises, or is capable of accessing, computer readable media comprising non-transitory computer-executable instructions which, when executed by at least one electronic processor, perform at least: receiving sequencing reads from a population of ribonucleic acid (RNA) molecules that each comprise polymeric nucleic acid tails and/or from derivative nucleic acid molecules thereof, wherein the RNA molecules comprise coding and non-coding RNAs, differentiating decorator sequence information from insert sequence information in the plurality of sequencing reads, determining orientations of coding RNA sequence information and non-coding RNA sequence information from the insert sequence information, removing or disregarding decorator sequence information from the insert sequence information to produce processed insert sequence information, and mapping the processed insert sequence information to a selected genomic transcriptome, thereby mapping the sequence information to the genomic transcriptome.

In another aspect, the disclosure provides a computer readable media comprising non-transitory computer-executable instructions which, when executed by at least one electronic processor, perform at least: receiving sequencing reads from a population of ribonucleic acid (RNA) molecules that each comprise polymeric nucleic acid tails and/or from derivative nucleic acid molecules thereof, wherein the RNA molecules comprise coding and non-coding RNAs, differentiating decorator sequence information from insert sequence information in the plurality of sequencing reads, and determining orientations of subsequences in the plurality of sequencing reads corresponding to the coding and non-coding RNAs using at least the insert sequence information.

In another aspect, the disclosure provides a computer readable media comprising non-transitory computer-executable instructions which, when executed by at least one electronic processor, perform at least: receiving sequencing reads from a population of ribonucleic acid (RNA) molecules that each comprise polymeric nucleic acid tails and/or from derivative nucleic acid molecules thereof, wherein the RNA molecules comprise coding and non-coding RNAs, differentiating decorator sequence information from insert sequence information in the plurality of sequencing reads, determining orientations of coding RNA sequence information and non-coding RNA sequence information from the insert sequence information, removing or disregarding decorator sequence information from the insert sequence information to produce processed insert sequence information, and mapping the processed insert sequence information to a selected genomic transcriptome, thereby mapping the sequence information to the genomic transcriptome.

Various optional features of the above embodiments include the following. The polymeric nucleic acid tail comprises a homopolymeric nucleic acid tail. The homopolymeric nucleic acid tail comprises a poly-A, poly-C, poly-U or poly-G nucleic acid tail. The decorator sequence information corresponds to nucleic acid sequences attached to the RNA molecules after obtaining the sample. The decorator sequence information corresponds to primer nucleic acid sequences, polymeric nucleic acid tail sequences, adapter nucleic acid sequences, or barcode nucleic acid sequences. The method comprises performing the attaching step of the polymeric nucleic acid tail and one or more polymerase chain reaction (PCR) steps in a single reaction container. The method further comprises size selecting the coding and non-coding RNAs in the sample to comprise longer (e.g., about 50 or more nucleotides in length) and shorter (e.g., about 50 or fewer nucleotides in length) RNA molecules of selected nucleotide lengths prior to obtaining the sequence information. The method further comprises separating the coding and non-coding RNAs from one or more other components of the sample prior to attaching the polymeric nucleic acid tail to the plurality of the non-coding RNAs in the sample. The other components comprise ribosomal RNAs (rRNAs), transfer RNAs (tRNAs), microRNAs (miRNAs), piwi RNAs (piRNAs), and any linear coding and non-coding RNAs present in the sample. The method further comprises determining relative amounts of the coding and non-coding RNAs in the sample. The method further comprises attaching one or more adapters to the RNA molecules that each comprise polymeric nucleic acid tails and/or to the derivative nucleic acid molecules thereof prior to obtaining the sequence information. The coding RNAs in the sample comprise poly-A nucleic acid tail sub-sequences prior to attaching the polymeric nucleic acid tail to the plurality of the non-coding RNAs in the sample. The coding RNAs comprise messenger RNAs (mRNAs). The coding RNAs are long RNAs that comprise a mean length that is greater than about 50, about 100, about 150, about 200, about 250, about 300, about 350, or more nucleotides. The non-coding RNAs comprise linear RNA molecules.

Various additional optional features of the above embodiments include the following. The non-coding RNAs comprise microRNAs (miRNAs). The non-coding RNAs are short RNAs that comprise a mean length that is less than about 50, about 40, about 30, about 20, or fewer nucleotides. The derivative nucleic acid molecules thereof comprise complementary deoxyribonucleic acid (DNA) molecules. The sample is obtained from a subject. The obtaining step comprises using at least one PCR-cDNA sequencing technique. The obtaining step comprises using at least one next generation sequencing technique. The next generation sequencing technique comprises at least one nanopore sequencing technique. The next generation sequencing technique comprises at least one single molecule sequencing technique. The sequence information comprises a plurality of sequencing reads and wherein the method further comprises determining orientations of coding RNA sequence information and non-coding RNA sequence information from the plurality of sequencing reads. The determining step comprises identifying sequencing reads corresponding to the coding and non-coding RNAs and identifying sequencing reads corresponding to complements or reverse complements of the coding and non-coding RNAs. The method further comprises mapping at least a portion of the sequence information to a genomic transcriptome. The method further comprises differentiating decorator sequence information from insert sequence information using the plurality of sequencing reads. The decorator sequence information corresponds to poly-A, poly-C, poly-U or poly-G nucleic acid tails of the coding and non-coding RNAs and/or to one or more adapters attached to the coding and non-coding RNAs using a non-templated nucleic acid polymerase. The method comprises determining the orientations of coding RNA sequence information and non-coding RNA sequence information and differentiating the decorator sequence information from the insert sequence information comprises combining a sequence alignment technique with an expression matching technique. The differentiating step comprises using at least one text view technique disclosed herein. The insert sequence information comprises the coding RNA sequence information and non-coding RNA sequence information. The method further comprises determining orientations of subsequences in the plurality of sequencing reads corresponding to the coding and non-coding RNAs using at least the insert sequence information, thereby the processing sequencing reads. The method further comprises re-orienting the subsequences in the plurality of sequencing reads corresponding to the coding and non-coding RNAs that are determined to be in a 3′ to 5′ orientation to a 5′ to 3′ orientation. The determining step comprises identifying whether the insert information is in a sense direction or in an antisense direction. The sequence information comprises a plurality of sequencing reads and wherein the method further comprising determining whether a given sequencing read is a well-formed sequencing read, a partial sequencing read, a naked sequencing read, or a fusion sequencing read.

### BRIEF DESCRIPTION OF THE DRAWINGS

The accompanying drawings (also “Figure” and “FIG.” herein), which are incorporated in and constitute a part of this specification, illustrate certain embodiments, and together with the written description, serve to explain certain principles of the methods, computer readable media, and systems disclosed herein. The description provided herein is better understood when read in conjunction with the accompanying drawings which are included by way of example and not by way of limitation. It will be understood that like reference numerals identify like components throughout the drawings, unless the context indicates otherwise. It will also be understood that some or all of the figures may be schematic representations for purposes of illustration and do not necessarily depict the actual relative sizes or locations of the elements shown.

FIG. 1 is a flow chart that schematically depicts exemplary method steps of substantially simultaneously detecting coding and non-coding linear ribonucleic acids (RNAs) in a sample according to some embodiments of the present disclosure.

FIG. 2 is a flow chart that schematically depicts exemplary method steps of processing sequencing reads according to some embodiments of the present disclosure.

FIG. 3 is a flow chart that schematically depicts exemplary method steps of mapping sequence information to a genomic transcriptome using a computer according to some embodiments of the present disclosure.

FIG. 4 is a flow chart that schematically depicts exemplary method steps of detecting non-coding linear ribonucleic acids (RNAs) in a sample according to some embodiments of the present disclosure.

FIG. 5 is a flow chart that schematically depicts exemplary method steps of substantially simultaneously detecting coding and non-coding linear ribonucleic acids (RNAs) in a sample according to some embodiments of the present disclosure.

FIG. 6 is a schematic diagram of an exemplary system suitable for use with certain embodiments of the present disclosure.

FIGS. 7A-C schematically show an exemplary PALS-NS experimental workflow (A), text-based model of a well-formed read (B) and custom bioinformatics pipeline (C). Dashed boxes indicate modifications to biochemical protocols, read models and bioinformatics pipeline.

FIG. 8 shows plots that illustrate the effect of molar RNA input on quality metrics of the sequencing: Adapter Dimers, Non-informative reads (=adapter dimers and non-mapped reads) and non-mapped inserts (out of the non-adapter dimer inserts). Points: represent individual sequencing run measures, “x” group averages. Plots are given in log-odds scale, with lower (negative) numbers indicating fewer adapter dimers, non-informative and non-mapped inserts.

FIG. 9 are plots that show representation of groups of RNA as a proportion of library depth in the 2×2 experiments; for each sub-library in each sequencing run (a total of eight sub-libraries per library) we calculated the representation of RNAs (counts/effective sub-library depth in log 10 scale) according to the group they belonged to: ERCC, HiM or LiM. We cross-classified results according to the RNA input used to construct the library (either ERCC or a mix of LIM, HIM and ERCC) and whether PAP had been included (PAP+) or not (sham poly-adenylation PAP-) after mapping against a sequence library that included the 92 ERCC RNAs and the 10 RNAs used in the mix (ERCC_miRmix). A sensitivity analysis was also performed by mapping against a database of the 92 ERCC and the entire miRbase. In the latter case, counts of RNAs not present in the mix (“NonSpikedMiRNAs”) were tabulated as a separate category.

FIGS. 10A and 10B are plots that show predicted library representation for a hypothetical depth of 10 million reads by insert type and read quality for the 2×2 experiments (A) and the Dilution Series (B). Sample Types included ERCC (without any microRNA input, “None”), or ERCC with spiked HIM and LiM (LiM+HIM+ERCC in A, LiM+HIM in B)

FIGS. 11A-11I are plots that show Generalized Additive Model (GAM) Negative Binomial estimates of the variation in sequence count over the 2×2 and dilution series experiment as a function of molar input of each RNA (A-G) and sequence length (H,I). In additional to the nine functionals, the GAM included a random effect for the (residual) bias factors for each distinct RNA included in these experiments (92 ERCC RNAs and 10 miRNAs for a total of 102 random effects).

FIGS. 12A-12D are Venn diagrams showing the overlap of individual RNAs detected in libraries constructed from a polyA enriched samples (Illumina), long RNA sequencing on a Nanopore device without polyadenylation i.e., ONT PAP(-), and PALS-NS for protein coding RNAs (A), long non-coding RNAs (B), microRNAs (C) and ribosomal RNAs (D).

FIGS. 13A-13D are plots that show clustering of counts (expressed as log 10 fractions of the library depth for each sequencing) for the two biological samples: Control Diet and High Fructose. To generate these figures, a multivariate clustering algorithm (Teigen) was applied to the three dimensional count data (PALS-NS, PAP (-) and Illumina) of the coding and non-coding RNAs from the two biological samples, for a total of four three dimensional clustering: non-coding RNAs in the Control Diet Sample (A), non-coding RNAs in the High Fructose sample (B), coding RNAs in the Control Diet Sample (C), coding RNAs in the High Fructose sample (D). Each subfigure shows the projection of the density in the three possible planes, the cluster indicator of each count and the centers of the clustering components. The thin dashed oblique lines show the direction of perfect (positive) correlation. The three long dashed horizontal lines are drawn at the linear threshold (upper two) and floor counts of the PALS-NS experiments.

FIGS. 14A-14D are plots that show the length of inserts mapping to the ERCC RNAs from the sham poly-adenylated samples (A), the 2×2 and ERCC polyadenylated samples from the DS (B), the LiM+HiM+ERCC samples in the DS (C) and the ERCCs spiked in the two biological samples (D). To generate the graph, ERCC RNAs were grouped together by length, ensuring there at least 4 RNAs per grouping category.

FIGS. 15A and 15B are plots that show length of inserts mapping to the human transcriptome in the biological samples from the PAP (-) Nanopore sequencing runs (A) and from the PALS-N protocol (B).

### Definitions

In order for the present disclosure to be more readily understood, certain terms are first defined below. Additional definitions for the following terms and other terms may be set forth through the specification. If a definition of a term set forth below is inconsistent with a definition in a patent application or issued patent that is incorporated by reference, the definition set forth in this application should be used to understand the meaning of the term.

As used in this specification and the appended claims, the singular forms “a,” “an,” and “the” include plural references unless the context clearly dictates otherwise. Thus, for example, a reference to “a method” includes one or more methods, and/or steps of the type described herein and/or which will become apparent to those persons of ordinary skill in the art upon reading this disclosure and so forth. It will also be appreciated that there is an implied “about” prior to the temperatures, concentrations, times, number of bases or base pairs, coverage, etc. discussed in the present disclosure, such that slight and insubstantial equivalents are within the scope of the present disclosure. In this application, the use of the singular includes the plural unless specifically stated otherwise. Also, the use of “comprise”, “comprises”, “comprising”, “contain”, “contains”, “containing”, “include”, “includes”, and “including” are not intended to be limiting.

It is also to be understood that the terminology used herein is for the purpose of describing particular embodiments only, and is not intended to be limiting. Further, unless defined otherwise, all technical and scientific terms used herein have the same meaning as commonly understood by one of ordinary skill in the art to which this disclosure pertains. In describing and claiming the methods, computer readable media, and systems, the following terminology, and grammatical variants thereof, will be used in accordance with the definitions set forth below.

About: As used herein, “about” or “approximately” or “substantially” as applied to one or more values or elements of interest, refers to a value or element that is similar to a stated reference value or element. In certain embodiments, the term “about” or “approximately” or “substantially” refers to a range of values or elements that falls within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value or element unless otherwise stated or otherwise evident from the context (except where such number would exceed 100% of a possible value or element).

Amplify: As used herein, “amplify” or “amplification” in the context of nucleic acids refers to the production of multiple copies of a polynucleotide, or a portion of the polynucleotide, typically starting from a small amount of the polynucleotide (e.g., a single polynucleotide molecule), where the amplification products or amplicons are generally detectable. Amplification of polynucleotides encompasses a variety of chemical and enzymatic processes.

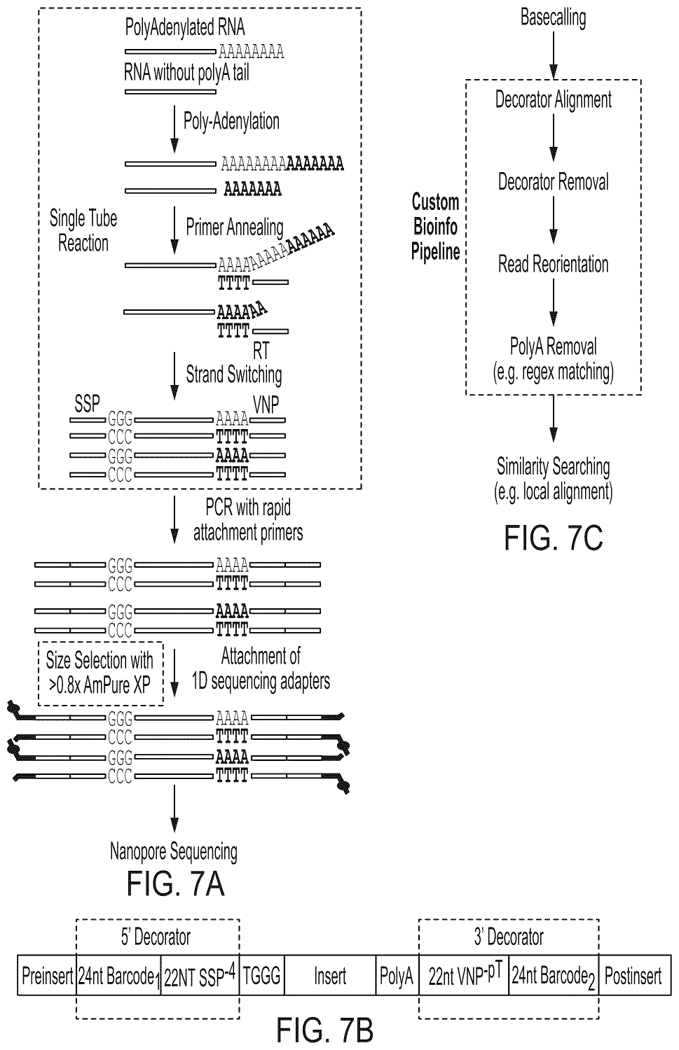

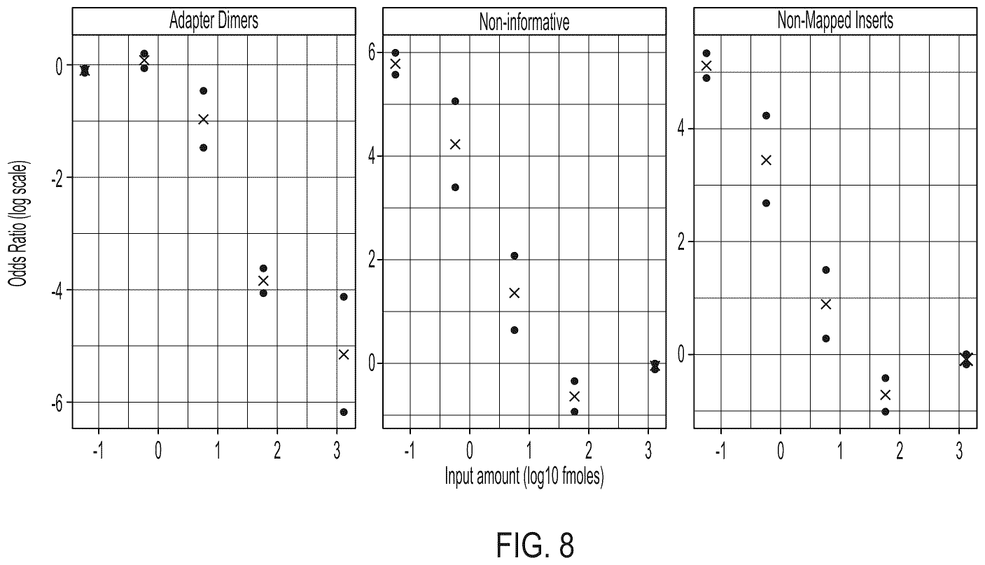

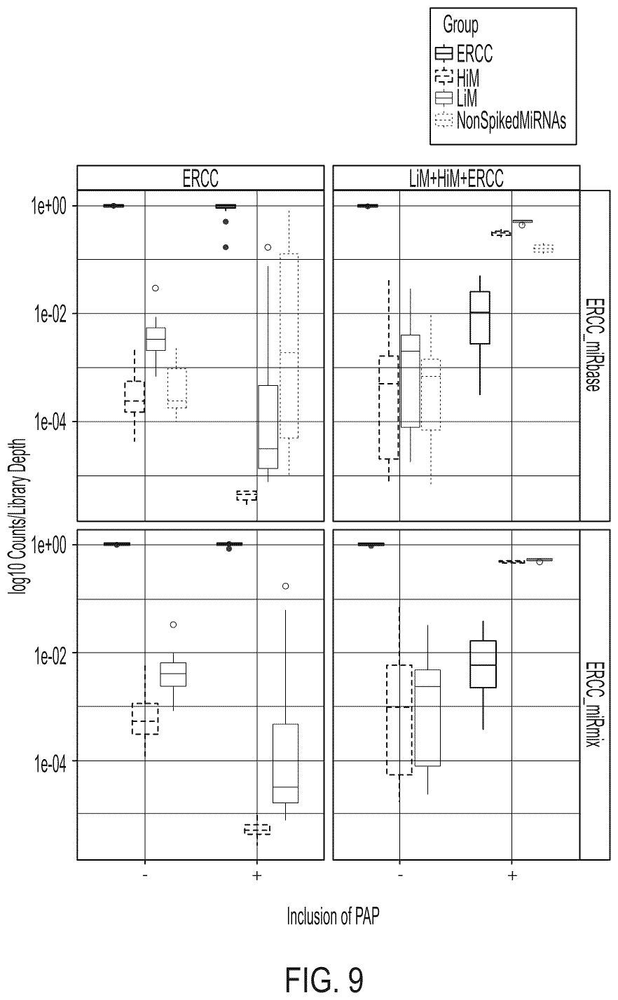

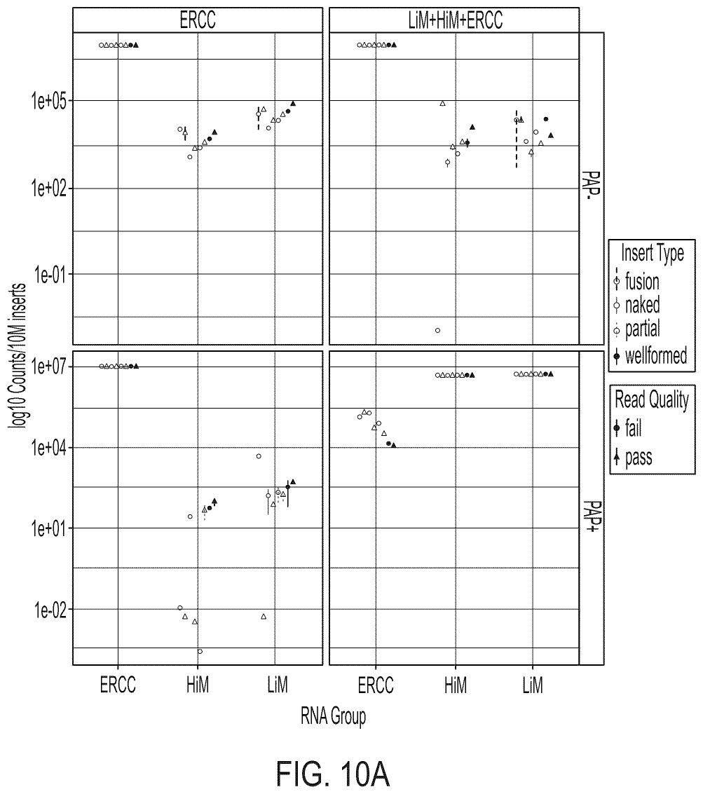

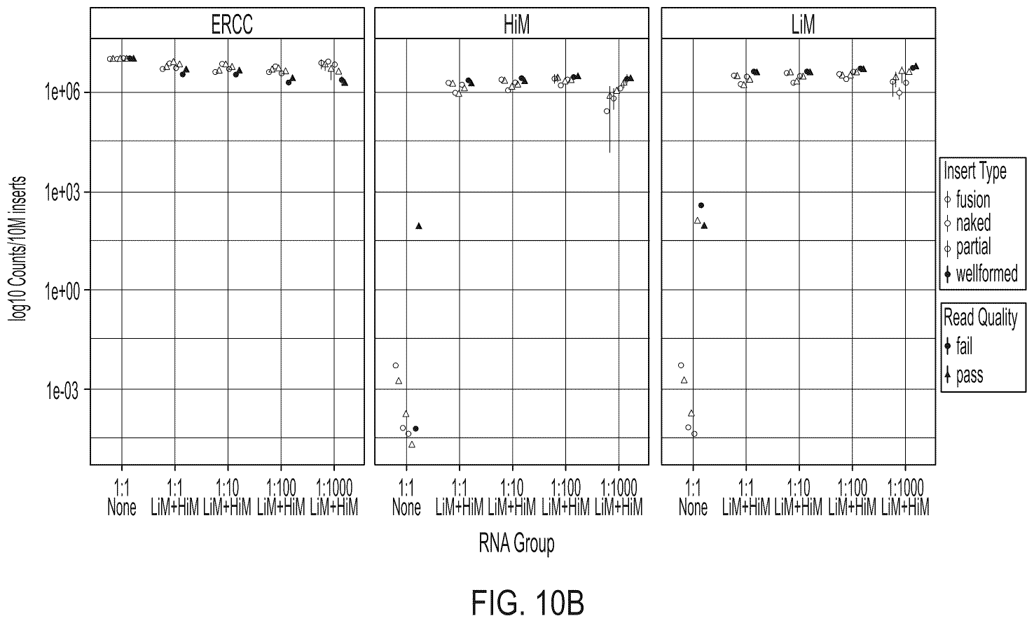

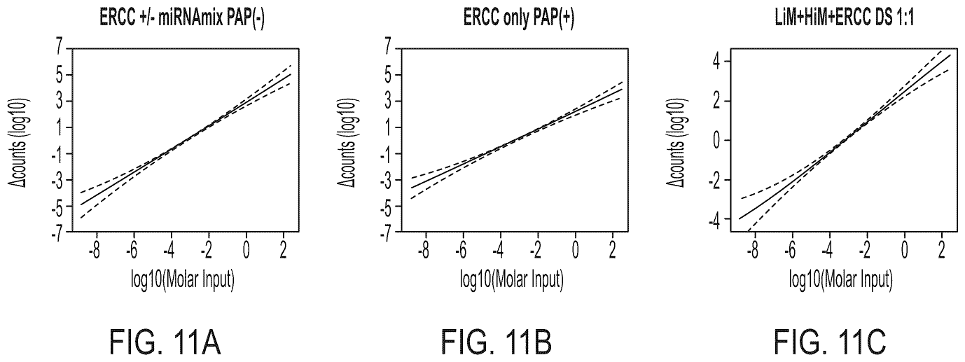

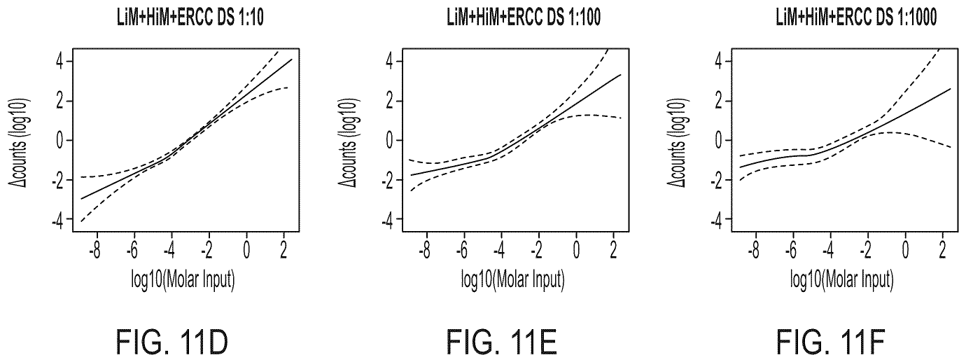

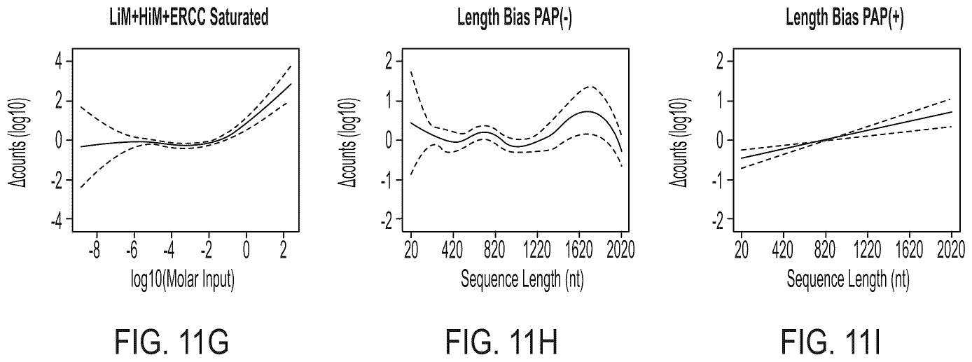

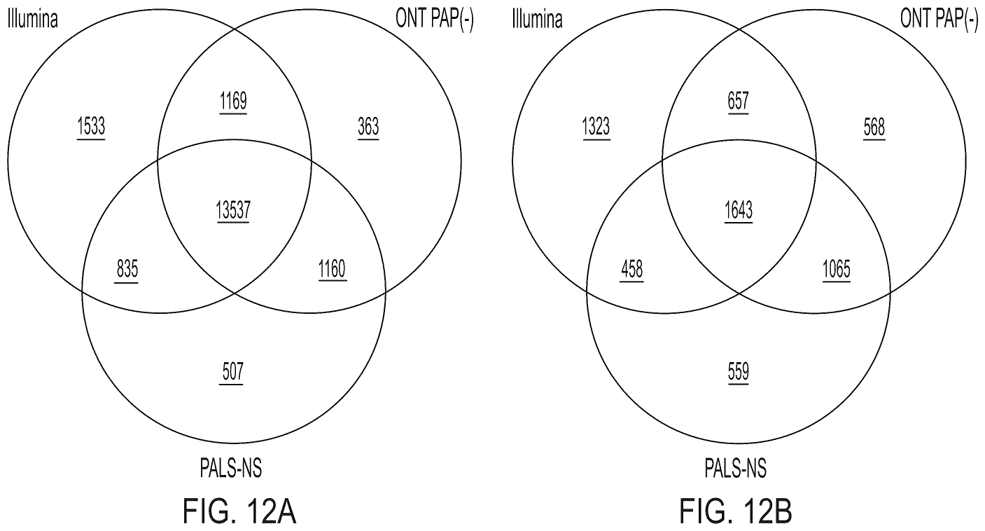

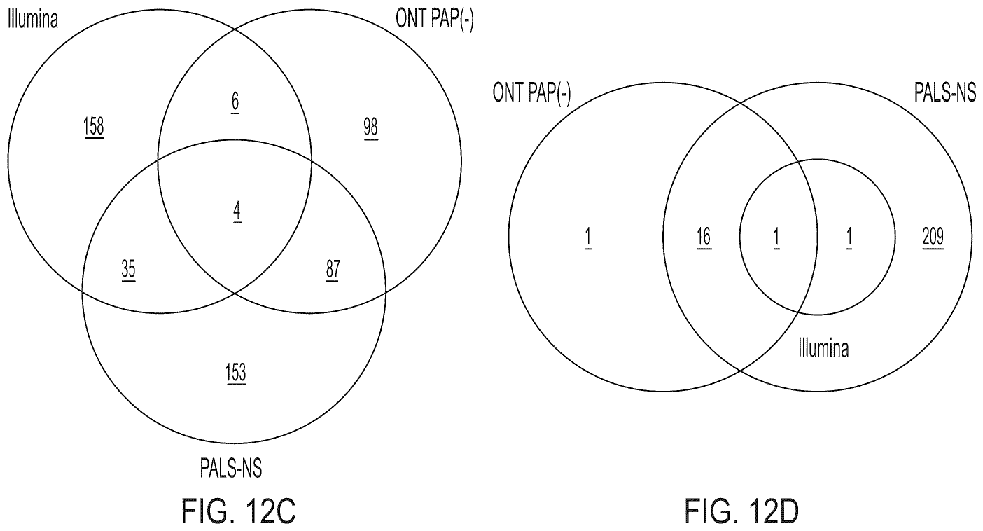

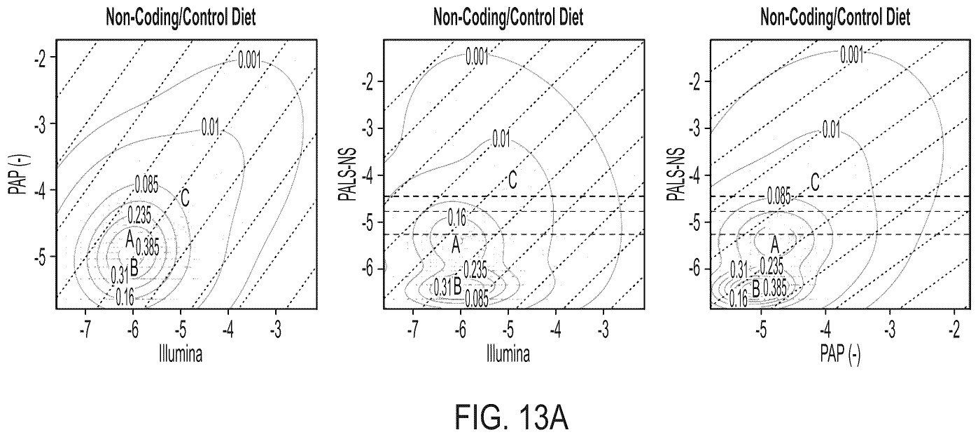

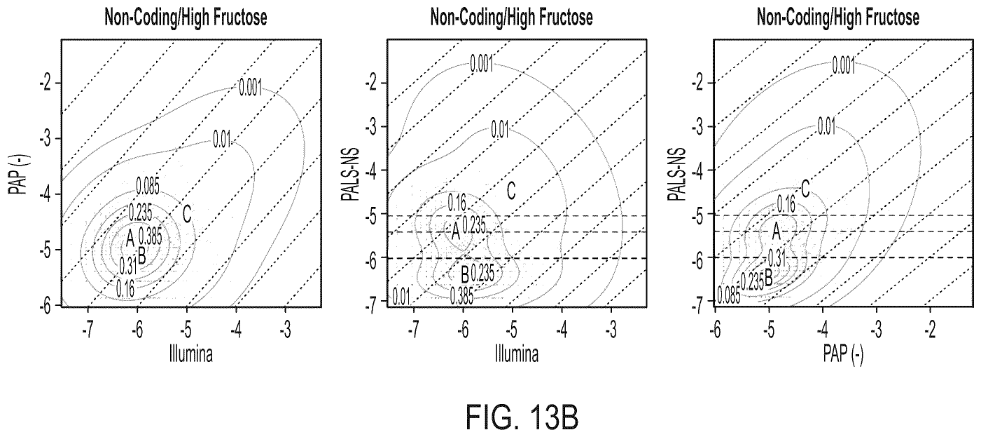

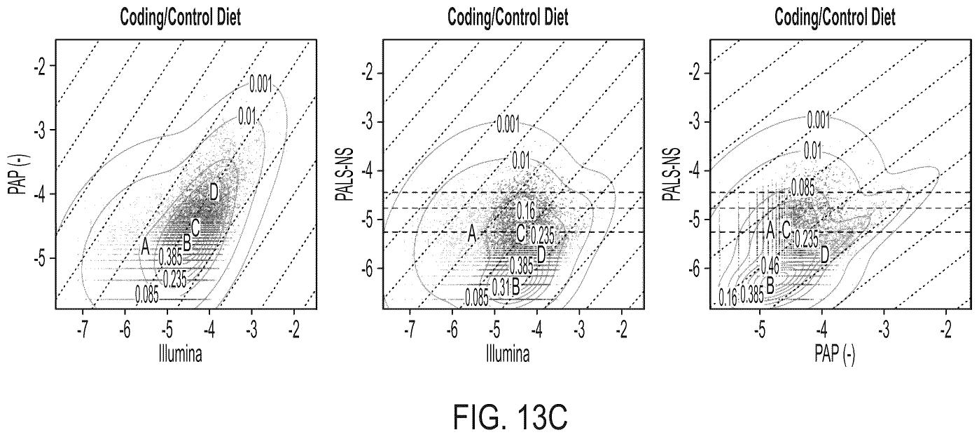

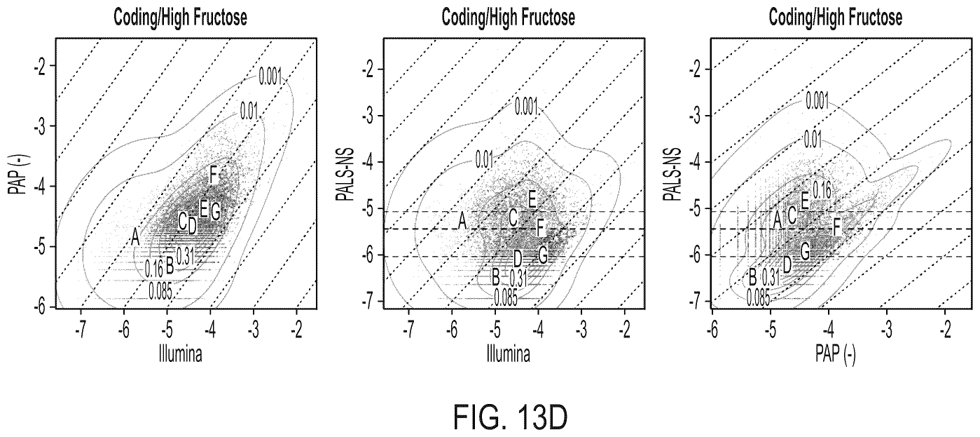

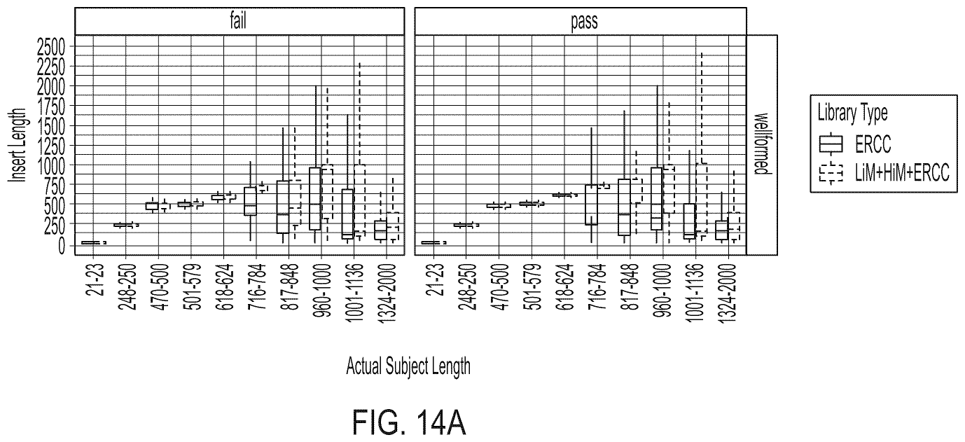

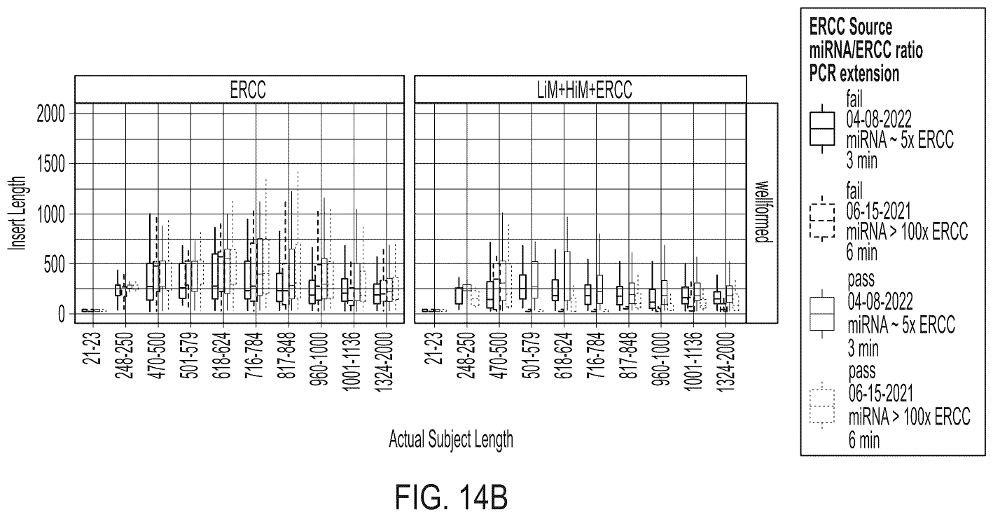

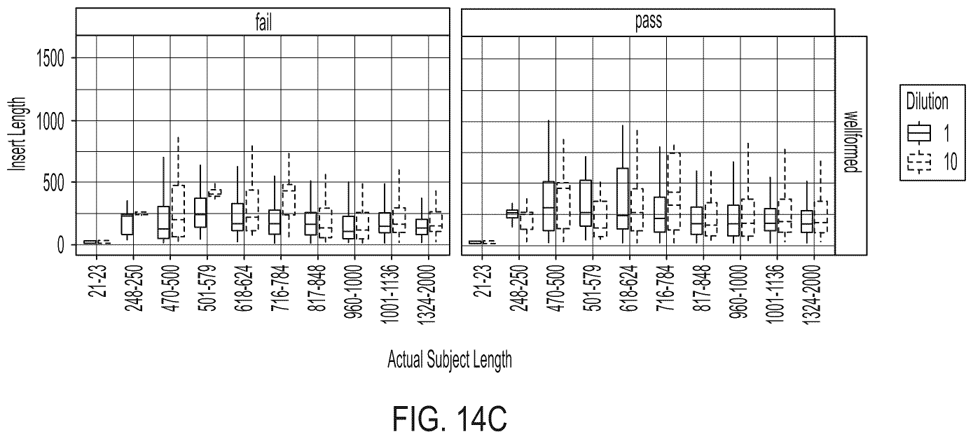

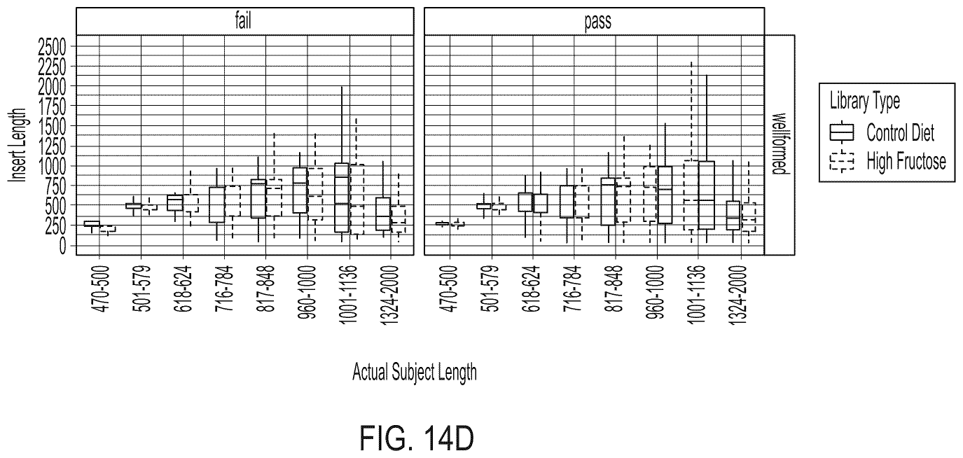

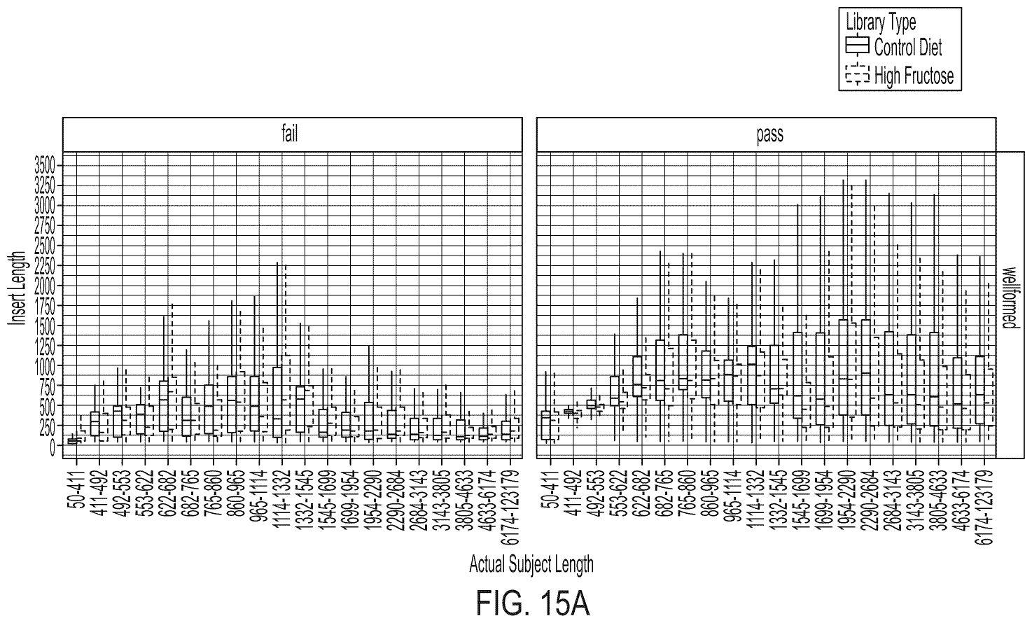

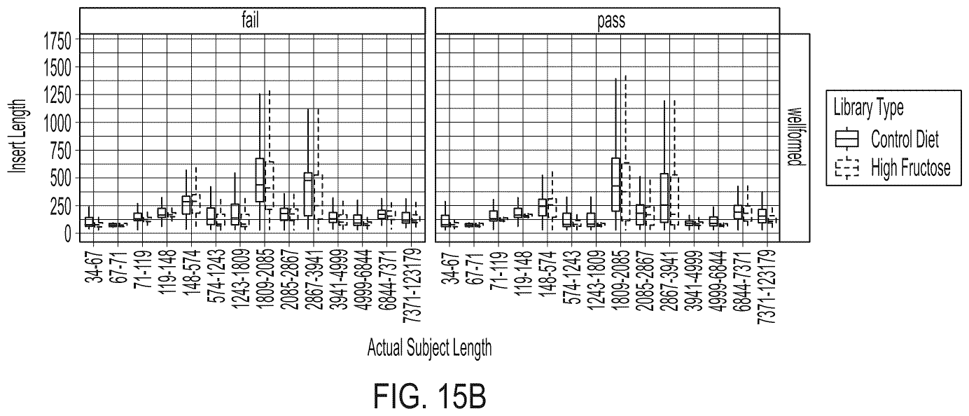

Decorator Sequence Information: As used herein, “decorator sequence information” refers to non-insert sequence information (e.g., non-target RNA or non-target derivative nucleic acid sequence information). Decorator sequence information can include, for example, sequence information corresponding to nucleic acid adapters, nucleic acid barcodes, nucleic acid tags, nucleic acid primer sequences, polymeric nucleic acid tails, or combinations thereof. In some embodiments, for example, a given target RNA insert or corresponding target derivative nucleic acid is flanked by 5′ and 3′ sequence decorators (e.g., derived from the primers of a PCR step used during a given library preparation process) and variable length pre-insert and post-insert sequences. As shown in FIG. 7B, in some embodiments, a 5′ decorator encompasses a 24nt barcode (Barcode₁) found in the middle of the reverse PCR primer and the 22 nucleotides of the SSP (sans the tetrabase TGGG, i.e., SSP⁻⁴), while 3′ decorator is composed of the VNP without its poly-T feature, i.e., VNP-PT and a 24nt Barcode₂ sequence.

Deoxyribonucleic Acid or Ribonucleic Acid: As used herein, “deoxyribonucleic acid” or “DNA” refers a natural or modified nucleotide which has a hydrogen group at the 2′-position of the sugar moiety. DNA typically includes a chain of nucleotides comprising deoxyribonucleosides that each comprise one of four types of nucleobases, namely, adenine (A), thymine (T), cytosine (C), and guanine (G). As used herein, “ribonucleic acid” or “RNA” refers to a natural or modified nucleotide which has a hydroxyl group at the 2′-position of the sugar moiety. RNA typically includes a chain of nucleotides comprising ribonucleosides that each comprise one of four types of nucleobases, namely, A, uracil (U), G, and C. As used herein, the term “nucleotide” refers to a natural nucleotide or a modified nucleotide. Certain pairs of nucleotides specifically bind to one another in a complementary fashion (called complementary base pairing). In DNA, adenine (A) pairs with thymine (T) and cytosine (C) pairs with guanine (G). In RNA, adenine (A) pairs with uracil (U) and cytosine (C) pairs with guanine (G). When a first nucleic acid strand binds to a second nucleic acid strand made up of nucleotides that are complementary to those in the first strand, the two strands bind to form a double strand. Examples of DNA or RNA, include genomic DNA, mitochondrial DNA, circulating DNA, cell-free DNA (cfDNA), cell-free RNA (cfRNA), coding RNA, non-coding RNA, small interfering RNA (siRNA), micro RNA (miRNA), circulating RNA (cRNA), transfer RNA (TRNA), ribosomal RNA (rRNA), small nucleolar RNA (snoRNA), Piwi-interacting RNA (piRNA), long non-coding RNA (lncRNA), short non-coding RNA (sncRNA), and/or fragments or hybrids thereof.

Derivative Nucleic Acid Molecule: As used herein, “derivative nucleic acid molecule” refers to a nucleic acid molecule that is produced based at least in part on another nucleic acid molecule. In some applications, for example, a complementary DNA (cDNA) molecule is a derivative nucleic acid molecule produced (e.g., reverse transcribed) from a corresponding RNA molecule. Other examples of derivative nucleic acid molecules, include amplicons produced in amplification reactions, such as polymerase chain (PCR) reactions.

Insert Sequence Information: As used herein, “insert sequence information” refers to non-decorator sequence information that comprises target RNA sequence information or target derivative nucleic acid sequence information.

Sequence Information: As used herein, “sequence information” in the context of nucleic acids denotes any information or data that is indicative of the order and identity of the nucleotide bases (e.g., adenine, guanine, cytosine, and thymine or uracil) in a molecule (e.g., a whole genome, whole transcriptome, exome, oligonucleotide, polynucleotide, or fragment) of a nucleic acid such as DNA or RNA. It should be understood that the present teachings contemplate sequence information obtained using all available varieties of techniques, platforms or technologies, including, but not limited to: nanopore-based systems, capillary electrophoresis, microarrays, ligation-based systems, polymerase-based systems, hybridization-based systems, direct or indirect nucleotide identification systems, pyrosequencing, ion- or pH-based detection systems, and electronic signature-based systems.

Next Generation Sequencing: As used herein, “next generation sequencing” or “NGS” refers to sequencing technologies having increased throughput as compared to traditional Sanger- and capillary electrophoresis-based approaches, for example, with the ability to generate hundreds of thousands of relatively small sequence reads at a time. Some examples of next generation sequencing techniques include, but are not limited to, nanopore sequencing, sequencing by synthesis, sequencing by ligation, and sequencing by hybridization.

Sample: As used herein, “sample” means anything capable of being analyzed by the methods and/or systems disclosed herein.

Sequencing: As used herein, “sequencing” refers to any of a number of technologies used to determine the sequence (e.g., the identity and order of monomer units) of a nucleic acid such as DNA or RNA. Exemplary sequencing methods include, but are not limited to, nanopore sequencing, targeted sequencing, single molecule real-time sequencing, solid-phase sequencing, high-throughput sequencing, massively parallel signature sequencing, emulsion PCR, exon or exome sequencing, intron sequencing, electron microscopy-based sequencing, panel sequencing, random shotgun sequencing, Sanger dideoxy termination sequencing, whole-genome sequencing, sequencing by hybridization, pyrosequencing, capillary electrophoresis, gel electrophoresis, duplex sequencing, cycle sequencing, single-base extension sequencing, transistor-mediated sequencing, direct sequencing, co-amplification at lower denaturation temperature-PCR (COLD-PCR), multiplex PCR, sequencing by reversible dye terminator, paired-end sequencing, near-term sequencing, exonuclease sequencing, sequencing by ligation, short-read sequencing, single-molecule sequencing, sequencing-by-synthesis, real-time sequencing, reverse-terminator sequencing, and a combination thereof. In some embodiments, sequencing can be performer by a gene analyzer such as, for example, gene analyzers commercially available from Oxford Nanopore Technologies (ONT), Pacific Biosciences, Inc., Illumina, Inc., or Applied Biosystems/Thermo Fisher Scientific, among many others.

Subject: As used herein, “subject” or “test subject” refers to an animal, such as a mammalian species (e.g., human) or avian (e.g., bird) species, or other organism, such as a plant. More specifically, a subject can be a vertebrate, e.g., a mammal such as a mouse, a primate, a simian or a human. Animals include farm animals (e.g., production cattle, dairy cattle, poultry, horses, pigs, and the like), sport animals, and companion animals (e.g., pets or support animals). A subject can be a healthy individual, an individual that has or is suspected of having a disease or a predisposition to the disease, or an individual that is in need of therapy or suspected of needing therapy. The terms “individual” or “patient” are intended to be interchangeable with “subject.”

### DETAILED DESCRIPTION

### Introduction

Sequencing of long coding RNAs informs about the abundance and the novelty in the transcriptome, while sequencing of short non-coding RNAs (e.g., microRNAs) or long non-coding RNAs informs about the epigenetic regulation of the transcriptome. Currently, each of these goals is addressed by separate sequencing experiments given the different physical characteristics of RNA species from biological samples. Sequencing of both short and long RNAs from the same experimental run has not been reported for long-read Nanopore sequencing to date and only recently has been achieved for short-read (Illumina) methods.

Accordingly, in some embodiments, the present disclosure provides library preparation methods capable of simultaneously profiling short and long RNA reads in the same library on nanopore platforms and also provides the relevant bioinformatics workflows to support the goals of RNA quantification. Using a variety of synthetic samples we demonstrate that the methods disclosed herein can simultaneously detect short and long RNAs in a manner that is linear over about five orders of magnitude for RNA abundance and about three orders of magnitude for RNA length. In biological samples the methods of the present disclosure are capable of profiling a wider variety of short and long non-coding RNAs when compared against the existing Smart-seq protocols for Illumina and nanopore sequencing. These and other attributes will be apparent upon a complete review of the present disclosure, including the accompanying figures.

### METHODS

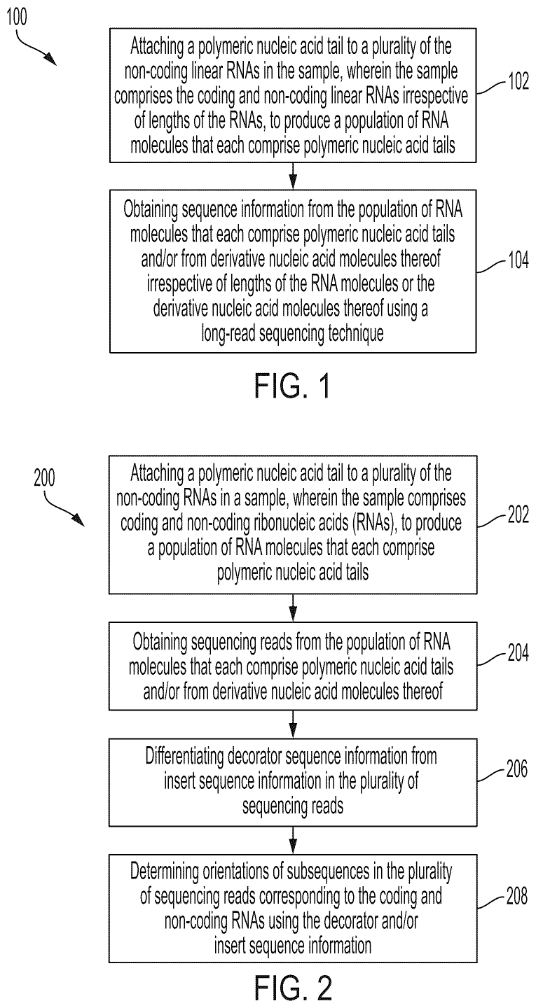

The present disclosure provides various methods for the simultaneous detection of short (e.g., about 50 or fewer nucleotides in length) and long (e.g., about 50 or more nucleotides in length) RNA molecules in samples and related library preparation processes. For example, FIG. 1 is a flow chart that schematically depicts exemplary method steps of substantially simultaneously detecting coding and non-coding linear ribonucleic acids (RNAs) in a sample according to some embodiments of the present disclosure. As shown, method 100 includes attaching a polymeric nucleic acid tail to a plurality of the non-coding linear RNAs in the sample to produce a population of RNA molecules that each comprise polymeric nucleic acid tails (step 102). The sample includes the coding and non-coding linear RNAs irrespective of lengths of the RNAs. Method 100 also includes obtaining sequence information from the population of RNA molecules that each comprise polymeric nucleic acid tails and/or from derivative nucleic acid molecules thereof irrespective of lengths of the RNA molecules or the derivative nucleic acid molecules thereof using a long-read sequencing technique, such as a nanopore sequencing procedure (step 104).

To further illustrate, FIG. 2 is a flow chart that schematically depicts exemplary method steps of processing sequencing reads according to some embodiments of the present disclosure. As shown, method 200 includes attaching a polymeric nucleic acid tail to a plurality of the non-coding RNAs in a sample, in which the sample comprises coding and non-coding ribonucleic acids (RNAs), to produce a population of RNA molecules that each comprise polymeric nucleic acid tails (step 202) and obtaining sequencing reads from the population of RNA molecules that each comprise polymeric nucleic acid tails and/or from derivative nucleic acid molecules thereof (step 204). In addition, method 200 also includes differentiating decorator sequence information from insert sequence information in the plurality of sequencing reads (step 206) and determining orientations of subsequences in the plurality of sequencing reads corresponding to the coding and non-coding RNAs using the decorator and/or insert sequence information (step 208).

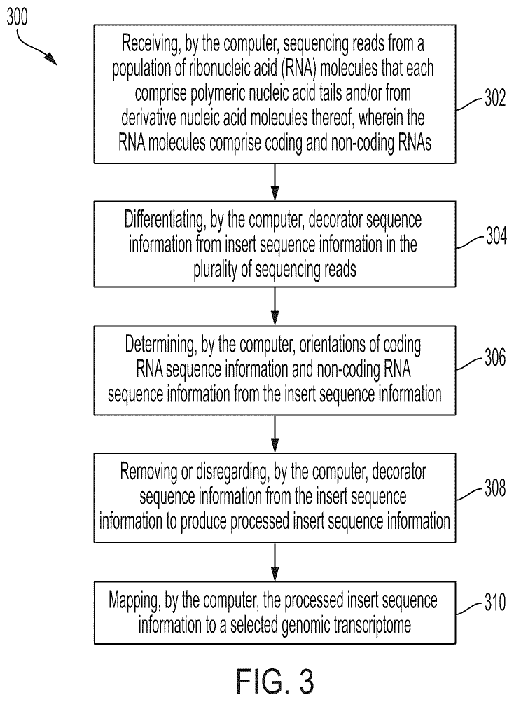

As an additional illustration, FIG. 3 is a flow chart that schematically depicts exemplary method steps of mapping sequence information to a genomic transcriptome using a computer according to some embodiments of the present disclosure. As shown, method 300 includes receiving, by the computer, sequencing reads from a population of ribonucleic acid (RNA) molecules that each comprise polymeric nucleic acid tails and/or from derivative nucleic acid molecules thereof, wherein the RNA molecules comprise coding and non-coding RNAs (step 302), differentiating, by the computer, decorator sequence information from insert sequence information in the plurality of sequencing reads (step 304), and determining, by the computer, orientations of coding RNA sequence information and non-coding RNA sequence information from the insert sequence information (step 306). In addition, method 300 also includes removing or disregarding, by the computer, decorator sequence information from the insert sequence information to produce processed insert sequence information (step 308) and mapping, by the computer, the processed insert sequence information to a selected genomic transcriptome (step 310).

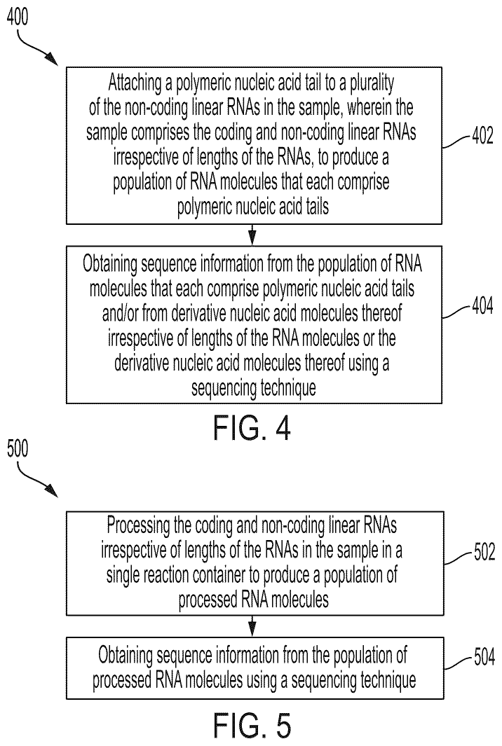

To further illustrate, FIG. 4 is a flow chart that schematically depicts exemplary method steps of detecting non-coding linear ribonucleic acids (RNAs) in a sample according to some embodiments of the present disclosure. As shown, method 400 includes attaching a polymeric nucleic acid tail to a plurality of the non-coding linear RNAs in the sample, wherein the sample comprises the coding and non-coding linear RNAs irrespective of lengths of the RNAs, to produce a population of RNA molecules that each comprise polymeric nucleic acid tails (step 402) and obtaining sequence information from the population of RNA molecules that each comprise polymeric nucleic acid tails and/or from derivative nucleic acid molecules thereof irrespective of lengths of the RNA molecules or the derivative nucleic acid molecules thereof using a sequencing technique, such as a nanopore sequencing procedure (step 404).

As another illustration, FIG. 5 is a flow chart that schematically depicts exemplary method steps of substantially simultaneously detecting coding and non-coding linear ribonucleic acids (RNAs) in a sample according to some embodiments of the present disclosure. As shown, method 500 includes processing the coding and non-coding linear RNAs irrespective of lengths of the RNAs in the sample in a single reaction container to produce a population of processed RNA molecules (step 502) and obtaining sequence information from the population of processed RNA molecules using a sequencing technique, such as a nanopore sequencing procedure (step 504).

In some embodiments, the polymeric nucleic acid tail comprises a homopolymeric nucleic acid tail, such as a poly-A, poly-C, poly-U or poly-G nucleic acid tail. The decorator sequence information corresponds to nucleic acid sequences attached to the RNA molecules after obtaining the sample. The decorator sequence information typically corresponds to primer nucleic acid sequences, polymeric nucleic acid tail sequences, adapter nucleic acid sequences, barcode nucleic acid sequences, or combinations and/or portions thereof. The methods of the present disclosure typically comprise performing the attaching step of the polymeric nucleic acid tail and one or more polymerase chain reaction (PCR) steps in a single reaction container.

In some embodiments, the methods disclosed herein further comprise size selecting the coding and non-coding RNAs in the sample to comprise longer (e.g., about 50 or more nucleotides in length) and shorter (e.g., about 50 or fewer nucleotides in length) RNA molecules of selected nucleotide lengths prior to obtaining the sequence information. In some embodiments, the methods of the present disclosure further comprise separating the coding and non-coding RNAs from one or more other components of the sample prior to attaching the polymeric nucleic acid tail to the plurality of the non-coding RNAs in the sample. In these embodiments, the other components may comprise ribosomal RNAs (rRNAs), transfer RNAs (tRNAs), microRNAs (miRNAs), piwi RNAs (piRNAs), and any linear coding and non-coding RNAs present in the sample. IN some embodiments, the method of the present disclosure further comprise determining relative amounts of the coding and non-coding RNAs in the sample.

In some embodiments, the methods disclosed herein further comprise attaching one or more adapters to the RNA molecules that each comprise polymeric nucleic acid tails and/or to the derivative nucleic acid molecules thereof prior to obtaining the sequence information. In some embodiments, the coding RNAs in the sample comprise poly-A nucleic acid tail sub-sequences prior to attaching the polymeric nucleic acid tail to the plurality of the non-coding RNAs in the sample. Typically, the coding RNAs comprise messenger RNAs (mRNAs). In some embodiments, the coding RNAs are long RNAs that comprise a mean length that is greater than about 50, about 100, about 150, about 200, about 250, about 300, about 350, or more nucleotides. Typically, the non-coding RNAs comprise linear RNA molecules. In some embodiments, the non-coding RNAs comprise microRNAs (miRNAs). The non-coding RNAs are generally short RNAs that comprise a mean length that is less than about 50, about 40, about 30, about 20, or fewer nucleotides. In some embodiments, derivative nucleic acid molecules comprise complementary deoxyribonucleic acid (cDNA) molecules.

In some embodiments, the sample is obtained from a subject, such as a human or other mammal. In some embodiments, the obtaining step comprises using at least one PCR-cDNA sequencing technique. In some embodiments, the obtaining step comprises using at least one next generation sequencing technique. In some embodiments, the next generation sequencing technique comprises at least one nanopore sequencing technique. In some embodiments, the next generation sequencing technique comprises at least one single molecule sequencing technique.

In some embodiments, the sequence information typically comprises a plurality of sequencing reads and in which the methods of the present disclosure further comprise determining orientations of coding RNA sequence information and non-coding RNA sequence information from the plurality of sequencing reads. In some embodiments, the determining step comprises identifying sequencing reads corresponding to the coding and non-coding RNAs and identifying sequencing reads corresponding to complements or reverse complements of the coding and non-coding RNAs. In some embodiments, the methods further comprise mapping at least a portion of the sequence information to a genomic transcriptome. In some embodiments, the methods of the present disclosure further comprise differentiating decorator sequence information from insert sequence information using the plurality of sequencing reads.

In some embodiments, the decorator sequence information corresponds to poly-A, poly-C, poly-U or poly-G nucleic acid tails of the coding and non-coding RNAs and/or to one or more adapters attached to the coding and non-coding RNAs using a non-templated nucleic acid polymerase. In some embodiments, the method disclosed herein comprise determining the orientations of coding RNA sequence information and non-coding RNA sequence information and differentiating the decorator sequence information from the insert sequence information comprises combining a sequence alignment technique with an expression matching technique. In some embodiments, the differentiating step comprises using at least one text view technique disclosed herein. Typically, the insert sequence information comprises the coding RNA sequence information and non-coding RNA sequence information. In some embodiments, the methods further comprise determining orientations of subsequences in the plurality of sequencing reads corresponding to the coding and non-coding RNAs using at least the insert sequence information, thereby the processing sequencing reads. In some embodiments, the methods of the present disclosure further comprise re-orienting the subsequences in the plurality of sequencing reads corresponding to the coding and non-coding RNAs that are determined to be in a 3′ to 5′ orientation to a 5′ to 3′ orientation. In some embodiments, the determining step comprises identifying whether the insert information is in a sense direction or in an antisense direction. The sequence information typically comprises a plurality of sequencing reads and in which the method further comprising determining whether a given sequencing read is a well-formed sequencing read, a partial sequencing read, a naked sequencing read, or a fusion sequencing read.

In these embodiments, the methods also typically include various sample or library preparation steps to prepare nucleic acids for sequencing. Many different sample preparation techniques are well-known to persons skilled in the art. Essentially any of those techniques are used, or adapted for use, in performing the methods described herein. For example, in addition to various purification steps to isolate nucleic acids from other components in a given sample, typical steps to prepare nucleic acids for sequencing include tagging nucleic acids with molecular identifiers or barcodes, adding adapters (e.g., which may include the barcodes), amplifying the nucleic acids one or more times, enriching for targeted segments of the nucleic acids (e.g., using various target capturing strategies, etc.), and/or the like. Exemplary library preparation processes are described further herein. Additional details regarding nucleic acid sample/library preparation are also described in, for example, van Dijk et al., Library preparation methods for next-generation sequencing: Tone down the bias, Experimental Cell Research, 322 (1): 12-20 (2014), Micic (Ed.), Sample Preparation Techniques for Soil, Plant, and Animal Samples (Springer Protocols Handbooks), 1^{st} Ed., Humana Press (2016), and Chiu, Next-Generation Sequencing and Sequence Data Analysis, Bentham Science Publishers (2018), which are each incorporated by reference in their entirety.

The methods disclosed herein are typically used to diagnose the presence of a disease, disorder, or condition, particularly cancer, in a subject, to characterize such a disease, disorder, or condition (e.g., to stage a given cancer, to determine the heterogeneity of a cancer, and the like), to monitor response to treatment, to evaluate the potential risk of developing a given disease, disorder, or condition, and/or to assess the prognosis of the disease, disorder, or condition. The methods disclosed herein are also optionally used for characterizing a specific form of cancer. Since cancers are often heterogeneous in both composition and staging, the data generated using the methods disclosed herein may allow for the characterization of specific sub-types of cancer to thereby assist with diagnosis and treatment selection. This information may also provide a subject or healthcare practitioner with clues regarding the prognosis of a specific type of cancer, and enable a subject and/or healthcare practitioner to adapt treatment options in accordance with the progress of the disease. Some cancers become more aggressive and genetically unstable as they progress. Other tumors remain benign, inactive or dormant.

In certain embodiments, tags providing molecular identifiers or barcodes are incorporated into or otherwise joined to adapters by chemical synthesis, ligation, or overlap extension PCR, among other methods. In some embodiments, the assignment of unique or non-unique identifiers, or molecular barcodes in reactions follows methods and utilizes systems described in, for example, U.S. patent application Ser. Nos. 20/030,152490, 20110160078, 20010053519, and U.S. Pat. Nos. 6,582,908, 7,537,898, and 9,598,731, which are each incorporated by reference.

Tags are linked to sample nucleic acids randomly or non-randomly. In some embodiments, tags are introduced at an expected ratio of identifiers (e.g., a combination of unique and/or non-unique barcodes) to microwells. For example, the identifiers may be loaded so that more than about 1, 2, 3, 4, 5, 6, 7, 8, 9, 10, 20, 50, 100, 500, 1000, 5000, 10000, 50,000, 100,000, 500,000, 1,000,000, 10,000,000, 50,000,000 or 1,000,000,000 identifiers are loaded per genome sample. In some embodiments, the identifiers are loaded so that less than about 2, 3, 4, 5, 6, 7, 8, 9, 10, 20, 50, 100, 500, 1000, 5000, 10000, 50,000, 100,000, 500,000, 1,000,000, 10,000,000, 50,000,000 or 1,000,000,000 identifiers are loaded per genome sample. In certain embodiments, the average number of identifiers loaded per sample genome is less than, or greater than, about 1, 2, 3, 4, 5, 6, 7, 8, 9, 10, 20, 50, 100, 500, 1000, 5000, 10000, 50,000, 100,000, 500,000, 1,000,000, 10,000,000, 50,000,000 or 1,000,000,000 identifiers per genome sample. The identifiers are generally unique and/or non-unique.

Sample nucleic acids flanked by adapters are typically amplified by PCR and other amplification methods using nucleic acid primers binding to primer binding sites in adapters flanking a DNA or RNA molecule to be amplified. In some embodiments, amplification methods involve cycles of extension, denaturation and annealing resulting from thermocycling, or can be isothermal as, for example, in transcription mediated amplification. Other exemplary amplification methods that are optionally utilized, include the ligase chain reaction, strand displacement amplification, nucleic acid sequence-based amplification, and self-sustained sequence-based replication, among other approaches.

One or more rounds of amplification cycles are generally applied to introduce molecular tags and/or sample indexes/tags to a nucleic acid molecule using conventional nucleic acid amplification methods. The amplifications are typically conducted in one or more reaction mixtures. Molecular tags and sample indexes/tags are optionally introduced simultaneously, or in any sequential order. In some embodiments, molecular tags and sample indexes/tags are introduced prior to and/or after sequence capturing steps are performed. In some embodiments, only the molecular tags are introduced prior to probe capturing and the sample indexes/tags are introduced after sequence capturing steps are performed. In certain embodiments, both the molecular tags and the sample indexes/tags are introduced prior to performing probe-based capturing steps. In some embodiments, the sample indexes/tags are introduced after sequence capturing steps are performed. Typically, sequence capturing protocols involve introducing a single-stranded nucleic acid molecule complementary to a targeted nucleic acid sequence, e.g., a coding sequence of a genomic region and mutation of such region associated with a cancer type. Typically, the amplification reactions generate a plurality of non-uniquely or uniquely tagged nucleic acid amplicons with molecular tags and sample indexes/tags at size ranging from about 200 nucleotides (nt) to about 700 nt, from 250 nt to about 350 nt, or from about 320 nt to about 550 nt. In some embodiments, the amplicons have a size of about 300 nt. In some embodiments, the amplicons have a size of about 500 nt.

Sample nucleic acids, optionally flanked by adapters, with or without prior amplification are generally subject to sequencing. Sequencing methods or commercially available formats that are optionally utilized include, for example, nanopore-based sequencing, Sanger sequencing, high-throughput sequencing, bisulfite sequencing, pyrosequencing, sequencing-by-synthesis, single-molecule sequencing, semiconductor sequencing, sequencing-by-ligation, sequencing-by-hybridization, RNA-Seq (Illumina), Digital Gene Expression (Helicos), next generation sequencing (NGS), Single Molecule Sequencing by Synthesis (SMSS) (Helicos), massively-parallel sequencing, Clonal Single Molecule Array (Solexa), shotgun sequencing, Ion Torrent, Oxford Nanopore, Roche Genia, Maxim-Gilbert sequencing, primer walking, sequencing using PacBio, SOLID, Ion Torrent, or nanopore platforms. Sequencing reactions can be performed in a variety of sample processing units, which may include multiple lanes, multiple channels, multiple wells, or other means of processing multiple sample sets substantially simultaneously. Sample processing units can also include multiple sample chambers to enable the processing of multiple runs simultaneously.

### Systems and Computer Readable Media

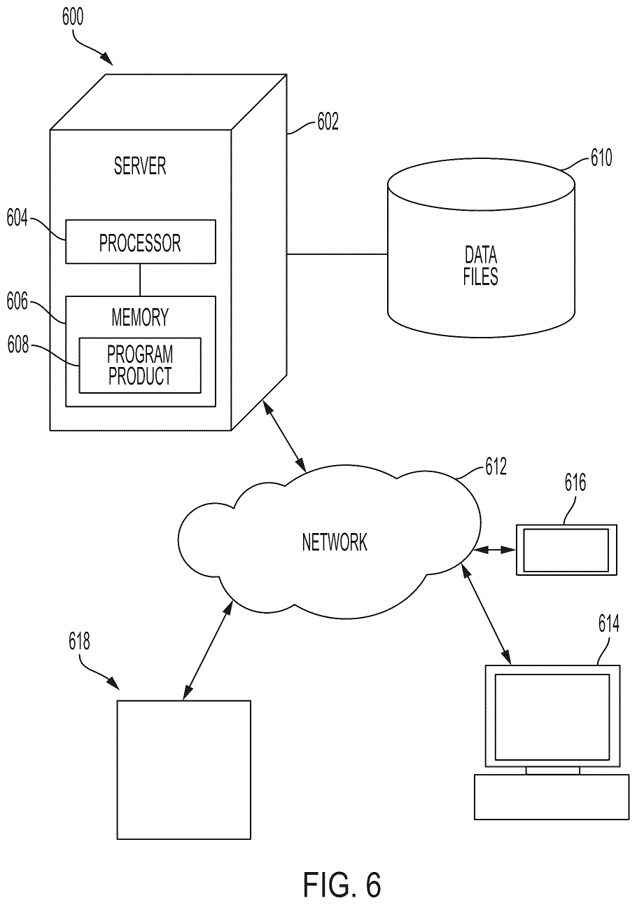

The present disclosure also provides various systems and computer program products or machine readable media. In some embodiments, for example, the methods described herein are optionally performed or facilitated at least in part using systems, distributed computing hardware and applications (e.g., cloud computing services), electronic communication networks, communication interfaces, computer program products, machine readable media, electronic storage media, software (e.g., machine-executable code or logic instructions) and/or the like. To illustrate, FIG. 6 provides a schematic diagram of an exemplary system suitable for use with implementing at least aspects of the methods disclosed in this application. As shown, system 600 includes at least one controller or computer, e.g., server 602 (e.g., a search engine server), which includes processor 604 and memory, storage device, or memory component 606, and one or more other communication devices 614 and 616 (e.g., client-side computer terminals, telephones, tablets, laptops, other mobile devices, etc.) positioned remote from and in communication with the remote server 602, through electronic communication network 612, such as the internet or other internetwork. Communication devices 614 and 616 typically include an electronic display (e.g., an internet enabled computer or the like) in communication with, e.g., server 602 computer over network 612 in which the electronic display comprises a user interface (e.g., a graphical user interface (GUI), a web-based user interface, and/or the like) for displaying results upon implementing the methods described herein. In certain embodiments, communication networks also encompass the physical transfer of data from one location to another, for example, using a hard drive, thumb drive, or other data storage mechanism. System 600 also includes program product 608 stored on a computer or machine readable medium, such as, for example, one or more of various types of memory, such as memory 606 of server 602, that is readable by the server 602, to facilitate, for example, a guided search application or other executable by one or more other communication devices, such as 614 (schematically shown as a desktop or personal computer) and 616 (schematically shown as a tablet computer). In some embodiments, system 600 optionally also includes at least one database server, such as, for example, server 610 associated with an online website having data stored thereon (e.g., sequence information, etc.) searchable either directly or through search engine server 602. System 600 optionally also includes one or more other servers positioned remotely from server 602, each of which are optionally associated with one or more database servers 610 located remotely or located local to each of the other servers. The other servers can beneficially provide service to geographically remote users and enhance geographically distributed operations.

As understood by those of ordinary skill in the art, memory 606 of the server 602 optionally includes volatile and/or nonvolatile memory including, for example, RAM, ROM, and magnetic or optical disks, among others. It is also understood by those of ordinary skill in the art that although illustrated as a single server, the illustrated configuration of server 602 is given only by way of example and that other types of servers or computers configured according to various other methodologies or architectures can also be used. Server 602 shown schematically in FIG. 6, represents a server or server cluster or server farm and is not limited to any individual physical server. The server site may be deployed as a server farm or server cluster managed by a server hosting provider. The number of servers and their architecture and configuration may be increased based on usage, demand and capacity requirements for the system 600. As also understood by those of ordinary skill in the art, other user communication devices 614 and 616 in these embodiments, for example, can be a laptop, desktop, tablet, personal digital assistant (PDA), cell phone, server, or other types of computers. As known and understood by those of ordinary skill in the art, network 612 can include an internet, intranet, a telecommunication network, an extranet, or world wide web of a plurality of computers/servers in communication with one or more other computers through a communication network, and/or portions of a local or other area network.

As further understood by those of ordinary skill in the art, exemplary program product or machine readable medium 608 is optionally in the form of microcode, programs, cloud computing format, routines, and/or symbolic languages that provide one or more sets of ordered operations that control the functioning of the hardware and direct its operation. Program product 608, according to an exemplary embodiment, also need not reside in its entirety in volatile memory, but can be selectively loaded, as necessary, according to various methodologies as known and understood by those of ordinary skill in the art.

As further understood by those of ordinary skill in the art, the term “computer-readable medium” or “machine-readable medium” refers to any medium that participates in providing instructions to a processor for execution. To illustrate, the term “computer-readable medium” or “machine-readable medium” encompasses distribution media, cloud computing formats, intermediate storage media, execution memory of a computer, and any other medium or device capable of storing program product 608 implementing the functionality or processes of various embodiments of the present disclosure, for example, for reading by a computer. A “computer-readable medium” or “machine-readable medium” may take many forms, including but not limited to, non-volatile media, volatile media, and transmission media. Non-volatile media includes, for example, optical or magnetic disks. Volatile media includes dynamic memory, such as the main memory of a given system. Transmission media includes coaxial cables, copper wire and fiber optics, including the wires that comprise a bus. Transmission media can also take the form of acoustic or light waves, such as those generated during radio wave and infrared data communications, among others. Exemplary forms of computer-readable media include a floppy disk, a flexible disk, hard disk, magnetic tape, a flash drive, or any other magnetic medium, a CD-ROM, any other optical medium, punch cards, paper tape, any other physical medium with patterns of holes, a RAM, a PROM, and EPROM, a FLASH-EPROM, any other memory chip or cartridge, a carrier wave, or any other medium from which a computer can read.

Program product 608 is optionally copied from the computer-readable medium to a hard disk or a similar intermediate storage medium. When program product 608, or portions thereof, are to be run, it is optionally loaded from their distribution medium, their intermediate storage medium, or the like into the execution memory of one or more computers, configuring the computer(s) to act in accordance with the functionality or method of various embodiments. All such operations are well known to those of ordinary skill in the art of, for example, computer systems.

To further illustrate, in certain embodiments, this application provides systems that include one or more processors, and one or more memory components in communication with the processor. The memory component typically includes one or more instructions that, when executed, cause the processor to provide information that causes sequence information, and/or the like to be displayed (e.g., via communication devices 614, 616, or the like) and/or receive information from other system components and/or from a system user (e.g., via communication devices 614, 616, or the like).

In some embodiments, program product 608 includes non-transitory computer-executable instructions which, when executed by electronic processor 604 perform at least: receiving sequencing reads from a population of ribonucleic acid (RNA) molecules that each comprise polymeric nucleic acid tails and/or from derivative nucleic acid molecules thereof, wherein the RNA molecules comprise coding and non-coding RNAs, differentiating decorator sequence information from insert sequence information in the plurality of sequencing reads, determining orientations of coding RNA sequence information and non-coding RNA sequence information from the insert sequence information, removing or disregarding decorator sequence information from the insert sequence information to produce processed insert sequence information, and mapping the processed insert sequence information to a selected genomic transcriptome. Additional computer readable media embodiments are described herein.

System 600 also typically includes additional system components that are configured to perform various aspects of the methods described herein. In some of these embodiments, one or more of these additional system components are positioned remote from and in communication with the remote server 602 through electronic communication network 612, whereas in other embodiments, one or more of these additional system components are positioned local, and in communication with server 602 (i.e., in the absence of electronic communication network 612) or directly with, for example, desktop computer 614.

In some embodiments, for example, additional system components include at least one nucleic acid sequencer 618 operably connected (directly or indirectly (e.g., via electronic communication network 612)) to controller 602. Nucleic acid sequencer 618 is configured to provide the sequence information from nucleic acids (e.g., ribonucleic acid (RNA) molecules) in samples from subjects. Essentially any type of nucleic acid sequencer can be adapted for use in these systems. For example, nucleic acid sequencer 618 is optionally configured to perform nanopore sequencing, single-molecule sequencing, semiconductor sequencing, bisulfite sequencing, pyrosequencing, sequencing-by-ligation, sequencing-by-hybridization, or other techniques on the nucleic acids to generate sequencing reads. Optionally, system 600 can also include other sub-system components, such as sample preparation components used for library preparation (e.g., attaching polymeric nucleic acid tails to a RNAs in a given sample), nucleic acid amplification components (e.g., thermal cyclers, etc.), material transfer component, or the like operably connected (directly or indirectly (e.g., via electronic communication network 612)) to controller 602.

### EXAMPLE: Poly-Adenylation Lengthening of Short RNAs for Simultaneous Short and Long Nanopore Sequencing (PALS-NS)

### Materials and Methods

Biochemical workflow/library preparation: In some embodiments, our protocol for the simultaneous detection of short and long RNAs polyadenylates all RNAs in a sample before using them as input to any Smart-seq protocol for long sequences (such as Oxford Nanopore's SQK-PCS109) that requires polyadenylated, poly(A)+, RNA. A major change introduced is the execution of the poly-adenylation in the same tube as the reverse transcription (RT) and template switching reactions, similar to the (Capture and Amplification by Tailing and Switching, CATS (22)/D-Plex Small RNA-seq) and Smart-seq-total workflows for Illumina sequencing. The remaining steps of the Smart-seq protocol are carried out without modifications: reverse transcription (RT) using a poly-T, VNP primer, addition of non-templated nucleotides (usually cytidines) strand switching via a strand switching primer (SSP) that contains a short ribo-nucleotide tail and PCR using universal primers that amplify between 5′ end of the SSP and 3′of the VNP. Finally, the amplified library is purified using AMPure XP (or equivalent) beads, the rapid sequencing adaptors are added, and the sample is loaded on the flow cell for sequencing. The brief duration of the poly-A tailing reaction in PALS-NS requires one further change to the Nanopore RNAseq protocols: size selection should be performed with 1.8× volumetric ratio of beads to library in order to retain both short and long cDNAs, instead of the usual 0.8×-1.0× ratio for long read sequencing.

Text model for Nanopore reads: The expected outcome of sequencing is a well-formed read containing a single insert that is derived from na RNA molecule in the original sample. In such reads the insert is flanked by the tetrabase TGGG (derived from 3′ of the SSP) and a poly-A tail at its 5′ and 3′ respectively. External to these features we find 5′ and 3′ sequence decorators (derived from the primers of the PCR during the library preparation) and variable length pre-insert and post-insert sequences. In the current iteration of the sequences used by ONT in their Smart-seq protocols, 5′ decorator encompasses a 24nt barcode (Barcode₁) found in the middle of the reverse PCR primer and the 22 nucleotides of the SSP (sans the tetrabase TGGG, i.e., SSP⁻⁴). The 3′ decorator is composed of the VNP without its poly-T feature, i.e., VNP-PT and a 24nt Barcode₂ sequence. At the time of this writing, ONT optionally uses the barcode sequences to multiplex samples (up to 12) for RNAseq; the preinsert and postinsert are derived from the 15nt long sequences flanking these barcodes.

Based on these structural considerations, we thus introduce a text-based model in which 5′ and 3′ decorator sequences play the role of opening “(” and closing “)” parentheses in natural language text. During sequencing, motor proteins are attached to both ends of the cDNA molecules in the library, so that molecules may be threaded through the nanopores from either 5′ or 3′ end. If the cDNA molecule is threaded from its 5′ end, it will be sequenced in 5′→3′ direction and we would read it as (TEXT), but if threaded from its 3′ end it will be sequenced in 3′→5′ direction we would read it as [rcTEXT]. In these expressions, rc stands for reverse complement, a bracket is the sequence of the decorators when the cDNA is sequenced in 3′→5′ direction, and TEXT is the sequence of interest comprised of the ACTG alphabet of DNA. The opening bracket “[” is thus the reverse complement of the closing parenthesis “)”, while the closing bracket “]” is the reverse complement of the opening parenthesis “(”. In our text-model, reads that appear to lack one or both decorators are classified as partial and naked respectively. It is also possible to have fusion reads which contain multiple parentheses and brackets as non-matching pairs e.g. (TEXT]. In such a case, each segment between consecutive parentheses or brackets is classified as a read.

Text Based Segmentation of PALS-NS files: In some embodiments, we use a text-based segmentation algorithm after basecalling the squiggle signal Nanopore files. The algorithm is composed of four steps:

1. Decorator alignment and filtering using an aligner of choice (in this work we used blastn).

2. Decorator removal by extending the decorator alignments to the entire length of the decorator and insert classification as one of the four types: well-formed, fusion, naked, partial. The first round of adapter dimer identification (stage 0 dimers, those whose insert is less than 4 nucleotides) takes place at this stage.

3. Identification of insert orientation based on the orientation of the surrounding brackets/parentheses and re-orientation of inserts whose sequencing direction has been unambiguously determined by the surrounding decorators, to be in the 5′→3′ direction.

4. Removal of poly-A tails from inserts e.g., via regular expression matching; inserts whose length is smaller than a second user defined threshold, e.g., ten nucleotides are classified as Stage 1 adapter dimers. Longer sequences are used for blastn database searches with an e-value threshold of 0.001 to determine similarity.

Model for Count Processing: Any given library is hypothesized to generate a set of mapped counts M₁, M₂, . . . , Mₘ belonging to m distinct RNA species, as well as a variable number of nonmapped counts (M₀) and adapter dimers (M₋₁). These counts may be modelled as draws from the multinomial distribution:

$${M}_{- 1} , {M}_{0} , {M}_{1} , … , {M}_{m} \vert N , {p}_{- 1}^{M} , {p}_{0}^{M} , {p}_{1}^{M} , … , {p}_{m}^{M} ∼ Multinomial ( N ; {p}_{- 1}^{M} , {p}_{0}^{M} , {p}_{1}^{M} , … , {p}_{m}^{M} ) \qquad ( Eq . 1 )$$

where N is the total number of inserts from the library (the library dep.), and

$${p}_{i}^{M} , i = - 1 , 0 , … ,$$

m is the fraction of the any given unique RNA species in the library. We can use the properties of the multinomial distribution to analyze:

1. The number of adapter dimers, since

$${M}_{- 1} \vert N , {p}_{- 1}^{M} ∼ Binomial ( N ; {p}_{- 1}^{M} )$$

2 The wasted library depth (the sum of adapter dimers and non-mapped reads), since

$${M}_{- 1} + {M}_{0} \vert N , {p}_{- 1}^{M} + {p}_{0}^{M} ∼ Binomial ( N ; {p}_{- 1}^{M} + {p}_{0}^{M} )$$

3. The number of non-mapped reads since

$${M}_{0} \vert N - {M}_{- 1} , {p}_{0}^{M} ∼ Binomial ( N - {M}_{- 1} ; {p}_{0}^{M} )$$

4. The RNA counts of interest M₁, . . . , Mₙ by conditioning on the effective library depth

$${N}_{eff} = N - {M}_{- 1} - {M}_{0} = {\sum }_{i = 1}^{m}$$

Mᵢ and the total probability of obtaining a useful read

$${p}_{*}^{M} = {\sum }_{k = 1}^{m} {p}_{k}^{M}$$

because

$${M}_{1} , … , {M}_{m} \vert {N}_{eff} , {p}_{1}^{M} , … , {p}_{m}^{M} ∼ Multinominal ( {N}_{eff} ; \frac{{p}_{1}^{M}}{{p}_{*}^{M}} , … , \frac{{p}_{m}^{M}}{{p}_{*}^{M}} ) \qquad ( Eq . 2 )$$

5. The RNA counts of interest

$${M}_{1}^{k} , … , {M}_{n}^{k}$$

from any sub-library, i.e., a subset of the entire library defined by shared characteristics, e.g. the type of insert, and the quality assigned to the corresponding read. This is a straightforward application of (Eq.2) with the counts and probabilities referring to the counts of reads with common features and the effective (sub-) library size is the total count for the particular sub-library.

The probabilities

$${p}_{i}^{M}$$

are proportional to this number of cDNA molecules loaded on the flow cell, which is proportional to the number of molecules of each RNA species in the sample (Xᵢ), and the efficiency of the steps of the library preparation. If we assume that the efficiency was the same for all RNAs, then we could simply set

$${p}_{i}^{M} ∝ {X}_{i} A ,$$

where A quantifies the common efficiency of library preparation. Since it is unlikely that this assumption holds true, we are content to write

$${p}_{i}^{M} ∝ {X}_{i} {Ae}^{{b}_{i}} ,$$

where bᵢ is a bias factor that quantifies the variability in library preparation. In this formulation, the factor A yields may be interpreted as a geometric average of the effects of library preparation on the RNA species present in the sample, and the factors bᵢ as deviations (“random effects”) from this average.

We now introduce a distributional approximation to the model in (Eq.2), that allows to replace the multinomial distribution with the product of independent Poisson random variates

$${M}_{1} , … , {M}_{m} \vert {N}_{eff} , {p}_{1}^{M} , … , {p}_{m}^{M} ≅ {\prod }_{i = 1}^{m} Poisson ( {N}_{eff} \times \frac{{p}_{i}^{M}}{{p}_{*}^{M}} ) \qquad ( Eq . 3 )$$

and impose a regression structure on the logarithm of the Poisson mean

$${μ}_{i} = {N}_{eff} \times \frac{{p}_{i}^{M}}{{p}_{*}^{M}} :$$

$$log {μ}_{l} \cdot = {λ}_{i} = log {N}_{eff} + log {X}_{i} + {b}_{i} + log \frac{A}{{p}_{*}^{M}} \qquad ( Eq . 4 )$$

In (Eq.4), the effective library depth, Neff, is the offset of the regression, and the parameter

$${μ}_{0} = log \frac{A}{{p}_{*}^{M}}$$

is the overall, grand mean. The Poisson models can be extended to account for overdispersion, and thus model additional sources of variation that would make RNA counts to be more variable than one would anticipate from Poissonian sampling. The simplest overdispersed model is the Negative Binomial one. In this work, we will be using the Poisson (or the binomial distribution) when the focus is on the performance of the sequencing itself (e.g., analyzing factors affecting the effective library depth), but switching to the Negative Binomial when interest lies in the expression of individual RNAs by aggregating counts over sub-libraries.

### Exploring Bias and Dynamic Range Compression in RNA Sequencing Via Mixed Poisson & Negative Binomial Models.

An additional complication for the analysis of counts is introduced by the size of the (effective) library depth: while one typically loads a few tens of femtomoles (˜10⁹), the sequenced libraries will have a depth ranging between 10⁵ (Flongle) to 10⁷ (Minlon). The impact of the limited depth relative to input is best understood by simulating (Eq.2) for various ranges of Xᵢ for an “average” RNA, i.e. one in which bᵢ=0. These simulations illustrate the compression of the dynamic range: RNAs which are present in fraction smaller than the threshold=Effective Library Depth/cDNA molecules in Library) will not generate any counts. The primary means of modelling this effect in a library with known inputs is to replace the logarithm in (Eq.4) by a more general function of the abundance and estimate this function from the data at hand. The simulations suggest a “stick-breaking” representation, i.e., a linear piecewise function that is constant below the detection threshold and a line with a slope of one for log Xᵢ above the threshold. The modeling task would then be to identify the threshold from counts of RNA species known inputs. However, the presence of noise around the detection threshold suggests that a smoother function (one that “curves”, rather than forming an acute angle around the threshold) would also be a viable option. The regression structure then becomes:

$${λ}_{i} = log {N}_{efff} + smo ( log {X}_{l} ) + {b}_{i} + {λ}_{0} \qquad ( Eq . 5 )$$

The threshold in this case, will be a “flat” area (a “floor”) over which the counts don't vary much, if at all, with changes in the input. The function smo(·) is given flexibly as a parameterized linear functional (e.g., a cubic or a thin plate spline). The parameters of the spline, which we denote as θₛ and the random effects corresponding to the bias factors may be estimated through penalized regression via Generalized Additive Models. The latter is a class of modeling tools which allow the data driven estimation of smooth functions and random effects from empirical data. If the input amount (Xᵢ) is known for each RNA, e.g. in the case of synthetic samples of known composition or exogenous spike-ins, then the bias factors b; could be estimated along with the smo(·) from the count data.

### Sources of RNA and Samples

Synthetic microRNAs: We selected 10 microRNAs for the sequencing experiments on the basis of previous work showing their relevance to the author's research field, kidney biology and pathophysiology. MicroRNAs were ordered as single stranded oligos from IDT from the sequences deposited in miRbase. One third of the RNAs terminated in ribo-adenines, and half of them included a ribo-adenine within 4 bases of their 3′ end. This was done to test the impact of sequencing errors by the Nanopore device due to the poly-A tails that will be attached to these short RNAs. Two of the microRNAs were closely related in sequence (200b-5p and 200c-5p) to test the impact of sequencing errors on the identification of microRNAs from the same family. Finally, one sequence (hsa-744-5p) has a ribo-guanine tetraplex; such sequences can form secondary structures which can complicate both synthesis and enzymatic reactions. Additionally, this microRNA has multiple ribo-adenines in its 3′ end, thus providing a special challenge to the proposed workflow. MicroRNAs were aliquoted in stock solutions of 100 μM in TE buffer provided by IDT (10 mM Tris, 0.1 mM EDTA, pH 7.5) and stored in −80° C. prior to sequencing. The ten microRNAs randomly allocated to two equimolar pools: a Hi(gh concentration) M(icroRNA)-HiM and a L(ow concentration) M(icroRNA)-LiM one. The final concentration of each RNA in the HiM pool, was double the concentration of each of the microRNAs in the LiM pool.

Synthetic long RNAs: A synthetic spike-mix (ERCC, Thermofisher, Catalog Number 4456740) was used as a source of long RNAs for the sequencing experiments and as a spike in control for the Nanopore experiments involving biological samples. ERCC is a common set of external, unlabeled, polyadenylated RNA controls that was developed by the External RNA Controls Consortium (ERCC) for the purpose of analyzing and controlling for sources of variation in transcriptomic workflows. These transcripts are designed to be 250 to 2,000 nucleotides (nt) in length, which mimic natural eukaryotic mRNAs. The 92 ERCC RNA control transcripts are divided into 4 different subgroups (A-D) of 23 transcripts each. These subgroups are mixed by the vendor to yield a moderate complexity synthetic mix of long transcripts with concentrations that span 6 orders of magnitude. The RNAs in the ERCC and the microRNAs selected, share common subsequences, i.e., half of the length of each short RNAs may be found as “words” inside the longer RNAs. Hence, despite the ERCC being unrelated to biologically derived long RNAs, fragments of the ERCC may be mistaken for short RNAs unless a high stringency sequence database search strategy is utilized. For our experiments we used two separate batches of ERCC (labelled as “A” and “B” in the results tables). The former was maintained in the −80° C. to simulate a biological sample under conditions of long-term storage, while the second source was stored as per vendor recommendations in the −20° C. and was used for the dilution and the PAP optimization experiments.

Construction of synthetic RNA samples (mixes): The microRNA and ERCC solutions were hand mixed together to generate the following samples:
a. Short RNA sample with a small amount of long, poly-adenylated long RNAs of the ERCC with equimolar mixes of the synthetic miRNAs. In these solutions the microRNAs were presented in a >100fold excess of the ERCC.
b. Long RNA samples which contained only the ERCC RNAs.
c. A more balanced mix of short and long RNAs in which the short RNAs were present in 5-fold excess over the long RNAs.
d. A dilution series 1:1, 1:10, 1:100, 1:1000 of the balanced mix. The preparations at higher dilution were used to simulate low input samples.

These solutions were used to explore the performance of PALS-NS to support a broad range of research agendas: a) poly-A depleted, e.g., short non-coding RNA or tRNA sequencing b) long coding RNA sequencing c) total RNA enriched in poly-adenylated sequences d) ultra-low input samples. RNA from these samples was used as input to the PALS-NS protocol.

Biological Samples: Total RNA was isolated from the jejunum of ten C57BI6/J mice from a series of experiments aimed at investigating the impact of different sources and amount of sugar on enteric and kidney physiology. Mice were housed in humidity-, temperature-, and light/dark-controlled rooms and cared for by trained individuals according to the Institutional Animal Care and Use Committees (IACUC) approved-protocols at the University of Cincinnati and the University of New Mexico. Mice were fed either a carbohydrate control or 60% fructose diet (Envigo, Indianapolis, IN) for 5 weeks. Mice were euthanized with an overdose of pentobarbital sodium and jejunum were harvested, cleaned of dietary material, snap frozen and stored at −80° C. Jejunal RNA was extracted using the TRI Reagent method (Molecular Research Center; Cincinnati, OH). RNA samples were stored at −80° C. until needed for Illumina sequencing. Two of the isolated samples (one from an animal fed a 60% fructose diet and one fed a carbohydrate control diet) were subjected to Nanopore sequencing using the proposed workflow and the unmodified PCR-cDNA Sequencing Protocol (SQK-PCS109) by Oxford Nanopore Technologies. The biological samples were used to provide an input to the protocol that reflects the composition of naturally occurring RNAs that could be used for library construction.

### Experimental Design

The effects of poly-adenylation in the context of the PALS-NS workflow were analyzed through a series of a 2×2 factorial design experiments utilizing the synthetic RNA samples constructed as detailed previously. In these experiments the two factors considered were: a) addition of short RNA mixes to the ERCC mix vs ERCC mix alone and b) Poly-Adenylation vs. Sham Poly-Adenylation. The latter was carried out by incorporating all the elements needed to carry out the poly-adenylation reaction (e.g., reaction buffer, ATP) except the PAP enzyme. In the first 2×2 experiment (“1^{st} 2×2”), the short RNAs were present in >100-fold excess of the ERCC RNA and the entire library was loaded on Minlon flow cells thus saturating the devices. The second of the 2×2 experiments (“2^{nd} 2×2”) was a replicate of the first experiment but only a fixed amount of library was loaded on the flow cells (with the amounts varying by flow cell type). These experiments were used to establish that the PALS-NS protocol can detect short RNAs and paved the way for a focused evaluation of the protocol on the lower capacity (Flongle) flow cells. Subsequently, we evaluated the linear dynamic range of PALS-NS with respect to changes in the molar input of the RNA in a dilution series (“DS”). To construct the DS, we diluted the synthetic mix of short and long RNAs 10-fold, 100-fold and 1000-fold and used these diluted samples as inputs for the library construction. These experiments were controlled by subjecting an ERCC mix to the PALS-NS protocol (only the higher dilution was tested).

### Pre-Sequencing Library Quantitation

Synthetic Samples: All synthetic samples were quantitated using High Sensitivity (HS) DNA assays on an Agilent 2100 Bioanalyzer system (Agilent Technologies, Santa Clara, CA). To remain within the assay's range of quantitation, libraries were diluted either 1:10 or 1:100 with ONT provided Elution Buffer prior to loading the chips. The bioanalyzer output was used to create working libraries of 100 femtomoles (for Minlon flow cells) or 26.12-50 femtomoles (for Flongle flow cells) of cDNA for loading onto the sequencers.

Biological Samples: Biological samples were quantitated with a Qubit 3.0 Fluorometer (Life Technologies) using the broad range RNA assay and rudimentary cDNA quality and size information was obtained from an Agilent 2100 Bioanalyzer with a Broad Range DNA Kit (Agilent, USA). For Qubit conversions from ug to picomoles cDNA, the following equation was used:

$$μ g cDNA \times \frac{pmol}{660 pg} \times \frac{1 {0}^{6}}{1 μ g} \times \frac{1}{N} = pmol cDNA$$

where 660 pg is the average molecular weight of a nucleotide pair, and ‘N’ is the predicted number of nucleotides. Upon visual inspection of the Bioanalyzer output, the typical length of the cDNA molecule was 500 bp, giving an estimated input of 200 fmoles to the sequencer.

### Sequencing

Nanopore sequencing: Sequencing experiments were done on two Mk1c devices and a single Mk1b device. The criterion for calling a read as low vs . . . high quality was a QC score of 8. Fast basecalling (Guppy) was used for all Minlon experiments and high accuracy basecalling for all Flongle experiments. Minlon cells were sequenced for 3 days and Flongle flow cells for 24 hrs, but the flow cells were exhausted before then (after approximately 1.5 days for Minlon cells and 9-10 hours for the Flongles). Experiments were run at ONT's default voltage and temperature settings of −180 mV and 35 degrees Celsius. All flow cells used were of the R9.4.1 chemistry except two flow cells used to sequence biological samples without a polyadenylation step that were of R10.4 chemistry.

Illumina Sequencing: The RNA-seq analysis of biological samples was performed by Novogene Bioinformatics Technology Co., Ltd (Beijing, China). Briefly, total RNA isolated from jejunum was subjected to quality control analysis using an Agilent 2100 Bioanalyzer with RNA 6000 Nano Kits (Agilent, USA). After poly A enrichment the samples were fragmented and reverse-transcribed to generate complementary DNA for sequencing. Libraries were sequenced on the HiSeq™ 2500 system (Illumina). Clean reads were aligned to mouse refence genome using Hisat2 v2.0.4.

### Database Mapping

Inserts from synthetic samples were mapped to two different databases of subject sequences: a) ERCC_miRmix, comprised of the 92 ERCC RNAs and the 10 microRNA sequences used to construct the synthetic mixes and b) ERCC_miRBase comprised of the 92 ERCC RNAs and the entire v22.0 mirBase of 48,885 sequences. When analyzing the latter database, we classified reads mapping to the ten microRNAs from different organisms to the human microRNA e.g. mmu-miR-192-5p reads were counted as hsa-miR-192-5p; all other RNAs in miRBase were classified as “NonSpikedRNAs”. To map the biological samples, we created two blast databases a) Mmusculus.39.cDNAncRNA that included all non-coding RNAs and cDNAs from the Genome Reference Consortium Mouse Build 39 and b) Mmusculus.39.cDNAncRNA_spike that enhanced the mouse database with the sequences of the ERCC spike-in mix. The package biomaRt was used to map the counts from the biological experiments to ensemble gene ids and eventually gene biotypes.

### Statistical Analysis & Software

A custom bioinformatics pipeline was developed to implement the text-based segmentation algorithm supporting PALS-NS. During segmentation, decorators, inserts and poly-A sequences were individually classified according to the type of the source read (well-formed, partial, fusion, naked) and the quality of the read (“pass” or “fail” as returned by Nanopore's MinKnow platform). During library mapping, the workflow counted the number of adapter dimers, non-mapped inserts and mapped inserts falling in these eight cross-classifications for each library and generated a text summary with various quality statistics for visual inspection. Result files were from these runs and metadata were loaded into sqlite3 using R's DBI interface. Custom R scripts were written to extract the information from the sqlite3 database for further analyses and deliver pilot implementations of the count processing algorithms, utilizing the GAM modeling package mgcv for random effects Poisson and Negative regressions. Insert characteristics were used to fit interaction models in which the effects of experimental factors, polyadenylation vs sham polyadenylation, synthetic RNA source and dilution level were allowed to vary in each by these eight characteristics. These models also allowed us to explore the hypothesis that the representation of distinct RNAs differed in these eight sub-libraries. If the composition of any of these sub-libraries differed from the one found in the gold-standard of the well-formed high-quality reads, then one should strongly consider removing the entire sub-library from further consideration. If on the other hand, the composition does not materially differ, then retaining the sub-library and basing the analyses on the entire set of counts without regard to sub-library type, will not only simplify analyses, but increase the statistical power of experiments based on PALS-NS. Model based cluster analysis with Student-t multivariate components (R package teigen) was used to visualize the concordance of libraries generated by different sequencing protocols from the two biological samples. A custom bioinformatics pipeline was developed to implement the text-based segmentation algorithm supporting PALS-NS. During segmentation, decorators, inserts and poly-A sequences were individually classified according to the type of the source read (well-formed, partial, fusion, naked) and the quality of the read (“pass” or “fail” as returned by Nanopore's MinKnow platform). During library mapping, the workflow counted the number of adapter dimers, non-mapped inserts and mapped inserts falling in these eight cross-classifications for each library and generated a text summary with various quality statistics for visual inspection. Result files were from these runs and metadata were loaded into sqlite3 using R's DBI interface (36). Custom R scripts were written to extract the information from the sqlite3 database for further analyses and deliver pilot implementations of the count processing algorithms, utilizing the GAM modeling package mgcv for random effects Poisson and Negative regressions. Insert characteristics were used to fit interaction models in which the effects of experimental factors, polyadenylation vs sham polyadenylation, synthetic RNA source and dilution level were allowed to vary in each by these eight characteristics. These models also allowed us to explore the hypothesis that the representation of distinct RNAs differed in these eight sub-libraries. If the composition of any of these sub-libraries differed from the one found in the gold-standard of the well-formed high-quality reads, then one should strongly consider removing the entire sub-library from further consideration. If on the other hand, the composition does not materially differ, then retaining the sub-library and basing the analyses on the entire set of counts without regard to sub-library type, will not only simplify analyses, but increase the statistical power of experiments based on PALS-NS. Model based cluster analysis with Student-t multivariate components (R package teigen) was used to visualize the concordance of libraries generated by different sequencing protocols from the two biological samples.

### Results

PALS-NS generates inserts of all types with high quality and variable poly-A tails. The sequencing conditions and overall counts are shown in. We undertook an Analysis of Variance to explore the impact of experimental factors, such as RNA input amount, PAP, flow cell type on the odds of obtaining adapter dimers, non-mapped reads and non-informative reads. The input amount was the most influential factor in these analyses. All three-quality metrics worsen (positive log-odds ratios) for inputs below ˜50 fmoles of RNA input. While adapter dimers continue to decline at higher molar inputs, there are diminishing returns in the other two metrics, largely driven by an increase in the proportion of reads that could not be mapped at the e-value cutoff chosen for these analyses. The vast majority of the experiments in the 2×2, DS as well as the biological samples generated a high number of high quality, well-formed reads (over than half); over 70% of the reads were either partial, or well-formed high quality ones. A linear regression analysis showed that the average poly-A tail (polyAinter0) from well-formed reads was 15 nucleotides long, while the interrupted poly-A tail types were longer by 5 and 10 nucleotides respectively. The polyAinter0 tails were mostly composed of adenines (98%), as were the polyAinter1 (92%) and polyAinter2 tails (81%). Poly-A tails from fusion and partial reads were shorter by 3 and 2 nucleotides respectively, but their adenine content was lower by 17% and 24% respectively compared to the well-formed reads.

TABLE 1
Sequencing statistics of synthetic libraries created

| | | | | | | | Library |
|---|---|---|---|---|---|---|---|
| | Library | | ERCC | Library | Sequencing | | Input^{†} |
| Experiment | Type | PAP | Source | Replicate | Replicate | Dilution | (fmoles) |
| 1^{st} 2 × 2 | LiM + HiM + | + | A | 1 | 1 | 1 | All |
| | ERCC | | | | | | |
| 2^{nd} 2 × 2 | LiM + HiM + | + | A | 2 | 1 | 1 | 100.00 |
| | ERCC | | | | | | |
| 1^{st} 2 × 2 | LiM + HiM + | − | A | 1 | 1 | 1 | All |
| | ERCC | | | | | | |
| 2^{nd} 2 × 2 | LiM + HiM + | − | A | 2 | 1 | 1 | 100.00 |
| | ERCC | | | | | | |
| 2^{nd} 2 × 2 | LiM + HiM + | − | A | 2 | 2 | 1 | 35.71 |
| | ERCC | | | | | | |
| 1^{st} 2 × 2 | ERCC | + | A | 1 | 1 | 1 | All |
| 2^{nd} 2 × 2 | ERCC | + | A | 2 | 2 | 1 | 34.72 |
| 1^{st} 2 × 2 | ERCC | − | A | 1 | 1 | 1 | All |
| 2^{nd} 2 × 2 | ERCC | − | A | 2 | 1 | 1 | 100.00 |
| 2^{nd} 2 × 2 | ERCC | − | A | 2 | 2 | 1 | 34.72 |
| DS | ERCC | + | B | 1 | 1 | 1 | 50.00 |
| DS | ERCC | + | B | 2 | 1 | 1 | 50.00 |
| DS | LiM + HiM + | + | B | 1 | 1 | 1 | 50.00 |
| | ERCC | | | | | | |
| DS | LiM + HiM + | + | B | 1 | 2 | 1 | 50.00 |
| | ERCC | | | | | | |
| DS | LiM + HiM + | + | B | 1 | 1 | 10 | 26.12 |
| | ERCC | | | | | | |
| DS | LiM + HiM + | + | B | 2 | 1 | 10 | 26.12 |
| | ERCC | | | | | | |
| DS | LiM + HiM + | + | B | 1 | 1 | 100 | 50.00 |
| | ERCC | | | | | | |
| DS | LiM + HiM + | + | B | 2 | 1 | 100 | 50.00 |
| | ERCC | | | | | | |
| DS | LiM + HiM + | + | B | 1 | 1 | 1000 | 50.00 |
| | ERCC | | | | | | |
| DS | LiM + HiM + | + | B | 2 | 1 | 1000 | 50.00 |
| | ERCC | | | | | | |

| | | | | Adapter | Noninformative | Mapped |
|---|---|---|---|---|---|---|
| | Flow | Unique | Unique | Dimers | Inserts | Inserts |
| Experiment | Cell | Reads | Inserts | N(%) | N(%) | N(%) |
| 1^{st} 2 × 2 | FLO- | 6575060 | 6660743 | 105908 | 3156686 | 3504057 |
| | MIN106 | | | (1.59) | (47.39) | (52.61) |
| 2^{nd} 2 × 2 | FLO- | 2801062 | 2994294 | 6265 | 1485668 | 1508626 |
| | MIN106 | | | (0.21) | (49.62) | (50.38) |
| 1^{st} 2 × 2 | FLO- | 7116660 | 7160292 | 33470 | 251827 | 6908465 |
| | MIN106 | | | (0.47) | (3.52) | (96.48) |
| 2^{nd} 2 × 2 | FLO- | 1044994 | 1051557 | 6831 | 75723 | 975834 |
| | MIN106* | | | (0.65) | (7.20) | (92.80) |
| 2^{nd} 2 × 2 | FLO- | 358255 | 367530 | 19566 | 51505 | 316025 |
| | FLG001 | | | (5.32) | (14.01) | (85.99) |
| 1^{st} 2 × 2 | FLO- | 9550475 | 9617510 | 142516 | 874487 | 8743023 |
| | MIN106 | | | (1.48) | (9.09) | (90.91) |
| 2^{nd} 2 × 2 | FLO- | 225335 | 245752 | 131694 | 245563 | 189 |
| | FLG001 | | | (53.59) | (99.92) | (0.08) |
| 1^{st} 2 × 2 | FLO- | 9272518 | 9325509 | 95731 | 664717 | 8660792 |
| | MIN106 | | | (1.03) | (7.13) | (92.87) |
| 2^{nd} 2 × 2 | FLO- | 1880918 | 1891459 | 14113 | 98648 | 1792811 |
| | MIN106* | | | (0.75) | (5.22) | (94.78) |
| 2^{nd} 2 × 2 | FLO- | 265439 | 267740 | 14895 | 37159 | 230581 |
| | FLG001 | | | (5.56) | (13.88) | (86.12) |
| DS | FLO- | 315945 | 316889 | 1303 | 20550 | 296339 |
| | FLG001 | | | (0.41) | (6.48) | (93.52) |
| DS | FLO- | 198666 | 209200 | 14235 | 57692 | 151508 |
| | FLG001 | | | (6.80) | (27.58) | (72.42) |
| DS | FLO- | 17307 | 18839 | 318 | 5274 | 13565 |
| | FLG001 | | | (1.69) | (28.00) | (72.00) |
| DS | FLO- | 650850 | 713759 | 18781 | 294390 | 419369 |
| | FLG001 | | | (2.63) | (41.25) | (58.75) |
| DS | FLO- | 99903 | 108326 | 19947 | 70427 | 37899 |
| | FLG001 | | | (18.41) | (65.01) | (34.99) |
| DS | FLO- | 137583 | 174903 | 67953 | 155390 | 19513 |
| | FLG001 | | | (38.85) | (88.84) | (11.16) |
| DS | FLO- | 549899 | 637985 | 352291 | 633907 | 4078 |
| | FLG001 | | | (55.22) | (99.36) | (0.64) |
| DS | FLO- | 632184 | 730683 | 357457 | 706693 | 23990 |
| | FLG001 | | | (48.92) | (96.72) | (3.28) |
| DS | FLO- | 189071 | 252980 | 118302 | 252341 | 639 |
| | FLG001 | | | (46.76) | (99.75) | (0.25) |
| DS | FLO- | 236026 | 279255 | 135012 | 278187 | 1068 |
| | FLG001 | | | (48.35) | (99.62) | (0.38) |

DS: Dilution Series,
ERCC: External RNA Controls Consortium reference RNA,
FLO-MIN106: Minlon Flow Cells,
FLO-FLG001: Flongle Flow Cells,
LiM: miRNAs input at lower ratio,
HiM: miRNAs input at higher ratio,
PAP: Poly A Polymerase,
*reused flow cell,
^{†}library input estimated via a high sensitivity Bioanalyzer chip (using appropriate dilutions if undiluted library runs failed to yield an estimate of molarity)

PALS-NS extends Nanopore long read sequencing to short non-coding RNAs. Representation of RNA groups in the 2×2 experiments is shown in. ERCC RNAs comprised the bulk (>99%) of all counts a) in the absence of a microRNAs in the sample (sample type ERCC, irrespective of the inclusion of PAP enzyme), and b) when a synthetic mix of microRNAs and ERCC (LIM+HIM+ERCC) was sequenced under sham poly-adenylation (PAP-). The detection rate of microRNAs in the samples which did not include PAP (˜0.1% of the effective library depth) is nearly identical to the expected false positive rate (e-value of 0.001) used during the blastn search. When the entire miRBase was used in database searches, the detection rate of the NonSpikedMiRNAs was of the same order of magnitude as that of the other microRNA groups in the sham poly-adenylated samples. The representation of the different RNA groups changed in the LIM+HIM+ERCC samples which were treated with PAP: the ERCC formed the minority of counts as expected from the molarity of the RNA mixes. Searching against the entire miRBase produced a small number of NonSpikedMiRNAs spurious reads (average 8.2% over all sub-libraries). The counts of the HIM and LiM groups decreased accordingly, suggesting that the spurious reads emanated from sequencing errors that led to the misclassification of short RNAs. Similar results were obtained in the dilution series experiment: in the absence of exogenous microRNA input, poly-adenylation does not generate a high rate of false positive short RNA reads, but when such RNAs are included in the RNA source, PALS-NS detects them irrespective of the amount of source RNA.

PALS-NS segmented inserts can be used to quantify RNA irrespective of insert type and sequencing quality of the source read. Well-formed reads typically accounted for ˜55% of all mappable reads, so we tested the hypothesis that the sub-library counts can be grouped together when quantifying RNAs and thus rescue the entire library for quantification. To do so, we fit Poisson regression models to the 2×2 experiment data and included all two way and higher order interactions among the following covariates: sample type (ERCC vs LiM+HIM+ERCC), PAP (sham vs . . . actual), read quality (pass vs fail), insert type (one of well-formed, pass, fail, fusion) and RNA group (ERCC, HIM, LiM and when mapping against miRbase NonSpikedMiRNAs). A similar model that included all two way and higher order interactions among dilution, sample type, read quality, insert type, RNA group was also fit to the dilution series. The analysis of variance table for the 2×2 experiments is shown in Table 1. The majority of the variance in counts is explained by the RNA Group (i.e. ERCC vs LiM vs HiM), design factors (Sample Type, inclusion of PAP) and interactions between the RNA, Sample Type and PAP. While statistically significant, the interaction terms between insert type, read quality and the experimental factors, explained far less of the variance in counts, and the impact of the latter was quantitatively very small. We illustrate the latter point by generating predictions of the model in Table 1 for a hypothetical library depth of 10 million inserts. The predicted counts for the RNA groups that were expected to be highly expressed (ERCC in all sham poly-adenylated samples and samples that did not include microRNA input, HiM+LiM in the poly-adenylated LiM+HIM+ERCC samples) were indistinguishable irrespective of the insert type and the quality of the read. On the other hand, predicted counts differed by insert type/read quality for RNA Groups that were either not expected to be present (e.g., the microRNAs in the non-polyadenylated samples) or anticipated to form only a small fraction of the library (ERCC counts in the LIM+HIM+ERCC libraries that was subjected to polyadenylation). Even in the latter case, the variation in the counts by insert type/read quality was rather small. Similar results were obtained for the dilution series, indicating that library representation did not materially differ according to insert type and read quality for low input samples.

TABLE 1
Analysis of variance for the effects of insert type, Read Quality, (RNA) Group
and design factors (Polyadenylation, Sample Type, i.e. ERCC vs HiM +
LiM + ERCC) in Poisson regressions for the counts from the 2 × 2 experiments

| | | | Residual | Residual | |
|---|---|---|---|---|---|
| | DF | Deviance | DF | Deviance | Pr(>Chi) |
| NULL | | | 257 | 63615714 | |
| Insert Type | 3 | 0 | 254 | 63615714 | 1 |
| Read Quality | 1 | 0 | 253 | 63615714 | 1 |
| PAP | 1 | 0 | 252 | 63615714 | 1 |
| Sample Type | 1 | 0 | 251 | 63615714 | 1 |
| Group | 2 | 36435133 | 249 | 27180581 | <2.2e−16 |
| Insert Insert Type:Read Quality | 3 | 0 | 246 | 27180581 | 1 |
| Insert Type:PAP | 3 | 0 | 243 | 27180581 | 1 |
| Read Quality:PAP | 1 | 0 | 242 | 27180581 | 0.9999513 |
| Insert Type:SampleType | 3 | 0 | 239 | 27180581 | 1 |
| Read Quality:SampleType | 1 | 0 | 238 | 27180581 | 0.9999513 |
| PAP:SampleType | 1 | 0 | 237 | 27180581 | 1 |
| Insert Type:Group | 6 | 969602 | 231 | 26210979 | <2.2e−16 |
| Read Quality:Group | 2 | 43554 | 229 | 26167425 | <2.2e−16 |
| PAP:Group | 2 | 8777999 | 227 | 17389426 | <2.2e−16 |
| SampleType:Group | 2 | 15920432 | 225 | 1468995 | <2.2e−16 |
| Insert Type:Read Quality:PAP | 3 | 17 | 222 | 1468978 | 0.0007149 |
| Insert Type:Read | 3 | 33 | 219 | 1468944 | 2.70E−07 |
| Quality:SampleType | | | | | |
| Insert Type:PAP:SampleType | 3 | 22637 | 216 | 1446307 | <2.2e−16 |
| Read Quality:PAP:SampleType | 1 | 1717 | 215 | 1444590 | <2.2e−16 |
| Insert Type:Read Quality:Group | 6 | 96 | 209 | 1444495 | <2.2e−16 |
| Insert Type:PAP:Group | 6 | 3309 | 203 | 1441186 | <2.2e−16 |
| Read Quality:PAP:Group | 2 | 191 | 201 | 1440994 | <2.2e−16 |
| Insert Type:SampleType:Group | 6 | 2476 | 195 | 1438518 | <2.2e−16 |
| Read | 2 | 706 | 193 | 1437812 | <2.2e−16 |
| Quality:SampleType:Group | | | | | |
| PAP:SampleType:Group | 2 | 1387521 | 191 | 50291 | <2.2e−16 |
| Insert Type:Read | 3 | 91 | 188 | 50201 | <2.2e−16 |
| Quality:PAP:SampleType | | | | | |
| Insert Type:Read | 6 | 125 | 182 | 50076 | <2.2e−16 |
| Quality:PAP:Group | | | | | |
| Insert Type:Read | 6 | 48 | 176 | 50028 | 1.08E−08 |
| Quality:SampleType:Group | | | | | |
| Insert | 6 | 17 | 170 | 50011 | 0.0084297 |
| Type:PAP:SampleType:Group | | | | | |
| Read | 2 | 63 | 168 | 49947 | 1.93E−14 |
| Quality:PAP:SampleType:Group | | | | | |
| Insert Type:Read | 6 | 11 | 162 | 49937 | 0.0926401 |
| Quality:PAP:SampleType:Group | | | | | |

The : indicate a statistical interaction; for example the Insert Type:PAP indicates the terms in the regression model that allow a different effect to be estimated for inserts of different type in the presence of polyadenylation.

PALS-NS quantifies RNAs over eight orders of magnitude of variation in source input while accounting for length and sequence dependence bias. Counts were linearly related to input amount in the absence of PAP, when ERCC was subjected to poly-adenylation and for the microRNA and ERCC mixture in the 1:1 dilution of the DS experiments. The results also demonstrate a progressive compression of the dynamic range as the effective library depth declined with successive dilutions in the DS. The saturated libraries in the 2×2 experiments demonstrate a more pronounced form of dynamic compression which was not the result of a decreased library depth (both libraries had >1.5 million mapped inserts) but was due to the 120-fold excess of microRNAs over ERCCs. In the absence of PAP, there was an inconsistent effect of sequence length on its representation; however there was a small, but definite linear length dependent bias in the presence of PAP: shorter sequences were under-represented relative to their input amount compared to longer sequences. Sequences with length of 20 nucleotides would be underrepresented by a factor of 1.2 log 10 ˜15.85 times relative to a sequence of 2,020 nucleotides that was present in the same amount as the short sequence in the original RNA sample. The estimated random effects (bias factors) from these analyses were determined. The estimate of the standard deviation of the random effects in log 10 scale was 0.482 (95% CI: 0.415-0.560), suggesting that 68%, 95%, 99.7% of all RNA counts will be within a factor ranging from 0.329-3.03, 0.108-9.26, 0.036-27.93 respectively relative to the value expected based on their input amount and their length. We also compared the magnitude of the bias factors of the short RNAs in the PAP+datasets against those obtained in a randomized/degenerate 5′ end 4N ligation based short RNA sequencing protocol. To carry out these analyses, we fit the Negative Binomial count model separately to the PAP+PALS-NS datasets and the publicly available data from the earlier report. There was no difference in the bias factors estimated for the common microRNAs obtained in these two very different RNAseq protocols (paired t-test for the difference in means p=0.88, Bonett-Seier test of variances for paired samples p=0.67).

PALS-NS extends the representation of non-coding RNAs in libraries from biological samples. RNA from a control mouse and one fed a high fructose diet were sequenced on an Illumina device, the unmodified SQKPCS109 workflow (denoted as ONT PAP(-) from this point onwards) and PALS-NS yielding libraries with a mapped library depth of 25,722,706/22,686,497 (Illumina), 434,467/717,012 (ONT PAP-) and 4,138,287/8,240,182 (PALS NS respectively when mapping against the Mmusculus.39.cDNAncRNA library). The number of mappable reads for PALS-NS was higher when the Mmusculus.39.cDNAncRNA_spike database was used for searches, i.e. 4,291, 187/8,525,805 because of the mapping of ERCC reads. The total number of reads obtained on the Nanopore devices were: ˜6.7M/13.8M (Control Diet Sample/High Fructose sample) for the PALS-NS runs and 0.96M/1.3M for the PAP (-) libraries respectively and more than 60% of inserts were mapped. All techniques detected a roughly similar proportion (68-74%) of unique protein coding transcripts and lncRNAs (13-15%). Other categories of non-coding RNAs (e.g., microRNAs, SnRNAs, ScaRNAs, SnoRNAs) were infrequently detected by Illumina and ONT PAP (-), but rose in frequency in the PALS-NS libraries. While all libraries detected the same protein coding RNAs, there was less overlap in the lncRNAs and much less in the microRNA and rRNA categories. Restricting attention to RNAs that had non-zero counts in at least one library, correlation was in general strong between libraries obtained by the same method (over 90%). Correlation was moderate between the Illumina and ONT PAP (-) libraries, and between ONT PAP (-) and PALS-NS (˜0.55-0.62) and weak between Illumina and PALS-NS (0.27-0.31). We then explored a) differences in the representation of various RNA species in the three library types and b) dynamic range compression and variably library depth as potential explanations for these variable correlations.

The representation of counts mapping to the Ensembl categories revealed some fundamental differences among the Illumina sequencing that used polyA enriched RNA, and the total RNA ONT PAP(-) and PALS-NS libraries. While 98% of the counts from the Illumina libraries mapped to protein coding RNAs, the latter comprised ˜84% of the total library depth in the ONT PAP(-) and only 30% of the PALS-NS libraries. Long non-coding RNAs were found in <1% of Illumina libraries, ˜3% of ONT PAP(-) but in ˜11% of the PALS-NS libraries. Other categories of non-coding RNAs of interest for epigenetics e.g., microRNAs, SnoRNAs, SnRNAs were detected at much higher percentages by PALS-NS. Of note, a significant number of PALS-NS reads mapped to ribosomal RNAs (42%) and mitochondrial transfer RNAs (10%) that were not detected in sizable proportions in the Illumina and ONT PAP (-) sequencing runs. Table 2 shows the statistical analysis of the differences in representation of (select) gene biotype categories. Compared to Illumina sequencing, ONT PAP(-) libraries had statistically significant increases in the representation of long non-coding RNAs (lncRNA), microRNAs, mitochondrial RNAs (Mt rRNA and Mt tRNA), ribozymes, small Cajal body RNAs (scaRNA), small nucleolar RNAs (snoRNA), small nuclear (snRNA) and mitochondrial RNAs. Compared to the ONT PAP(-), PALS-NS increased the representation of all non-coding RNAs (except Mt RNA) and decreased as a result the representation of protein coding RNAs.

TABLE 2
Statistical analysis of representation of select gene biotype categories in ONT
PAP (−) and PALS-NS libraries versus the same categories in Illumina libraries

| | Gene Biotype | | | | | |
|---|---|---|---|---|---|---|
| | ONT PAP (−) vs Illumina | | | PALS-NS vs ONT PAP (−) | | |
| | RR | 95% CI | p-value | RR | 95% CI | p-value |
| protein | 0.853 | 0.547-1.328 | 0.481 | 0.358 | 0.230-0.558 | <0.001 |
| coding | | | | | | |
| lncRNA | 5.203 | 3.340-8.107 | <0.001 | 3.519 | 2.259-5.482 | <0.001 |
| miRNA | 114.617 | 73.337-179.133 | <0.001 | 4.360 | 2.796-6.798 | <0.001 |
| misc RNA | 465.770 | 250.327-866.632 | <0.001 | 98.037 | 61.807-155.505 | <0.001 |
| Mt rRNA | 31.964 | 20.510-49.815 | <0.001 | 0.186 | 0.119-0.290 | <0.001 |
| Mt tRNA | 85.042 | 54.518-132.654 | <0.001 | 11.298 | 7.249-17.608 | <0.001 |
| Ribozyme | 98.336 | 36.730-263.268 | <0.001 | 50.425 | 26.121-97.342 | <0.001 |
| rRNA | 116067.975 | 40619.876-331654.747 | <0.001 | 41.778 | 26.808-65.108 | <0.001 |
| scaRNA | 4.620 | 2.472-8.634 | <0.001 | 9.256 | 5.022-17.061 | <0.001 |
| scRNA | 3252.378 | 99.079-106762.804 | <0.001 | 3.240 | 1.754-5.983 | <0.001 |
| snoRNA | 35.412 | 22.593-55.504 | <0.001 | 8.197 | 5.244-12.813 | <0.001 |
| snRNA | 13.944 | 8.716-22.306 | <0.001 | 14.480 | 9.108-23.022 | <0.001 |

lncRNA: long non-coding RNA,
miRNA: microRNA,
Mt rRNA: mitochondrial rRNA,
Mt tRNA: Mitochondrial tRNA,
scaRNA: small Cajal body RNA,
scRNA: small nuclear RNA,
snoRNA: small nucleolar RNA,
RR: Relative Ratio,
CI: Confidence Interval
Relative Ratios computed on the basis of a Negative Binomial GAM that included all gene biotype categories.
Model used a random effect smoother that incorporated gene biotype and sequencing protocol library, as well as random effects at the individual library level.

The effects of dynamic range compression in the biological samples was determined. The floor of detection, the highest count that demarcates the area in which counts don't vary appreciably by input was visually estimated to be 8 and 16 for the Control Diet and High Fructose libraries. Between the floor and the counts obtained for the most abundant spiked-in RNA, the curve relating molar input to counts appears to undergo two changes in linear slope: the more proximal (to the floor) appears to be at counts of 74 and 155 for the High Fructose and Control Diet libraries, and the terminal one at counts 32 and 74 respectively. To explore the role of the dynamic threshold, we applied a model-based clustering analysis to the counts from non-coding and coding RNAs from the two experiments. The non-coding RNA counts from the 2 samples could be resolved as a cluster of three components. Component A is the cluster of the non-coding RNAs that were not reliably captured by either the Illumina or the PAP (-) ONT library. This cluster appears as a “vertical” ellipse that extends mostly above the floor of PALS-NS for both biological samples, but its projection on the Illumina-PAP (-) plane is oriented along the diagonal because the (low) counts from these two protocols are concordant. Component B is an “horizontal” ellipse of non-coding RNAs with very low counts in the PALS-NS experiments, but with counts that ranged over two orders of magnitude in the Illumina and ONT PAP (-) experiments. These are RNAs whose counts were compressed because of the reduced effective library depth for the complexity of the PALS-NS samples. The correlation of the RNAs mapping to the components A and B is very small as the relevant components are oriented vertically and horizontally respectively. Finally component C includes RNAs that were sequenced above the linear thresholds in both biological samples. The relevant component is oriented along the bottom left-top right direction in the PAP(-)-Illumina and PALS-NS-PAP (-) plane implying a weak positive correlation, but along the top left-bottom right direction in the Illumina-PALS-NS plane implying a negative correlation. Both dynamic range compression, as implied by the component B, and detection of RNAs by PALS-NS that are poorly detectable by the other sequencing protocols (component A) underline the poor correlation among the counts of non-coding RNAs obtainable by PALS-NS, PAP (-) and Illumina. Clustering analysis resolved the coding RNA counts to 4 (Control Diet sample) and 7 (High Fructose sample) components. Coding RNAs captured well by PALS-NS and PAP (-), but not by Illumina map to component A. Components B and D (Control Diet) and B, D and G (High Fructose Diet) are captured by all three sequencing and the relevant components are oriented along a bottom left-top right diagonal indicating a positive and strong correlation. The remaining components (C in the Control Diet, C, E, F in the High Fructose Diet) are RNAs whose counts are highly correlated between the Illumina and PAP (-) libraries, as evidenced by their orientation along the bottom left-top right axis. However, the projection of these components to the 2 PALS-NS planes map at or below the linear thresholds established by the ERCC spike-in analysis, but towards the middle of the Illumina and bottom of the PAP (-) range of counts; these are RNAs whose expression in the PALS-NS libraries was compressed. In summary, the moderate to poor correlation between PALS-NS and either PAP (-) or Illumina is explained by expansion of its repertoire to non-coding RNAs and compression of the dynamic range for the coding RNAs by the over-representation of ribosomal and other non-coding RNAs in the PALS-NS samples.

Length of transcripts sequenced by PALS-NS varies according to the amount of short RNAs present in the sample. In sham-polyadenylated synthetic samples, the length of the ERCC inserts was highly reproducibly and closely tracked the known length of the ERCC irrespective of read quality, or the presence of microRNAs, up to lengths of 784 nucleotides; the length of inserts mapping to longer ERCC RNAs fell below the theoretical length after that point, and only short inserts (below 1000 bases) were recovered for the longest ERCC RNAs. When the synthetic samples were subjected to polyadenylation, the same overall pattern was observed, but there was substantial variability in the insert length within the same length category in the absence of microRNAs, irrespective of the ERCC source and operational PCR parameters, panel ERCC). Inclusion of microRNAs at lower (5×) vs . . . higher (>100×) amount relative to the ERCC resulted in longer ERCC mapped inserts irrespective of read quality, panel LiM+HIM+ERCC). Variation in insert read length did not improve when the synthetic mix of microRNAs and ERCC was diluted; in both the 2×2 experiments and the DS, the ERCC inserts were shorter than the reference sequence length. The median ERCC insert length in the biological samples appeared to linearly increase in tandem with the reference length up to 784 nucleotides but declined for longer ERCC sequences. Similarly, the length of inserts mapping to the human transcriptome in the biological samples was longer in the PAP (-) experiments compared to the PALS-NS runs.

### DISCUSSION

In this example we present a complete solution for the simultaneous profiling of short and long RNAs (either coding and non-coding) from the same library preparation that is sequenced on Nanopore's device platform. The solution comprises a single tube, single step addition of a homopolymeric tail to the Smart-seq protocol for Nanopore sequencing and a custom bioinformatics pipeline that utilizes a text segmentation model to extract the inserts (RNA sequences in the original sample) from the sequencing reads. Using a variety of synthetic mixes and biological samples we document that the proposed method exhibits exquisite linear dynamic range and will effectively profile both short and long RNAs via Nanopore sequencing. This result, which is due to the innovative combination of biochemistry, a dedicated bioinformatic approach and the single molecule detection capabilities of the Nanopore platform, opens unique opportunities for system biology and biomarker discovery.

Relation of PALS-NS to previous approaches for short RNA quantification: SMART-seq protocols are ubiquitous since the introduction of the SMART full-length cDNA library construction method more than twenty years ago. SMART based protocols utilize a poly-T oligonucleotide to hybridize to the poly-A tail of RNAs, followed by reverse transcription and template switching to synthesize full length first strands. Therefore, RNAs such as microRNAs or transfer RNAs that lack a poly-A tail cannot be analyzed with this technique. Such RNAs can be sequenced via alternative ligation and circularization protocols with the former being the default approach to microRNA sequencing. On the other hand, poly-adenylation tagging has been one of the major approaches to quantifying microRNAs by PCR methods using universal DNA or Locked Nucleic Acid primers. Our PAP approach is unique by i) clearly separating the PAP and RT reactions in time, but not in space, ii) avoiding exposure of poly-adenylated RNAs to high temperatures in the presence of magnesium from the PAP reaction buffer that could promote hydrolysis of longer RNAs, iii) moving the entire product to the RT step after cold inactivation and iv) utilizing long, rather than short read RNA sequencing. Our approach avoids setting up networks of competitive reaction between the RT and the PAP as both would try to access 3′ end of the RNAs in the reaction solution. The combination of these technical innovations probably accounts for the high sensitivity of detection of RNAs but also the low sequence and length dependent bias we documented in our analysis. Sequential PAP protocols like ours that execute the PAP and RT steps in a single tube have been reported in the literature previously. However, these protocols though target either qPCR or the Illumina sequencing platforms e.g. CATS/D-Plex, Smart-seq-total and thus fail to reap the benefits of long reads, such as reduced length dependent bias and even bias against non-coding RNAs as we discuss below. However, the unique features of Nanopore sequencing requires a text model-based segmentation algorithm to leverage the capabilities of PALS-NS reads which we also discuss below.

Text model-based segmentation facilitates quantitative analyses of Nanopore Libraries: PALS-NS and other PAP based extensions of SMART-seq protocols for short-read platforms generate a highly structured read. This ideal structure is observed in most, but not all, reads obtained from a single Nanopore experiment as we showed in this work. The variations from the ideal can manifest in various ways, i.e., truncated sequences (naked or partial reads), or fusion/chimeric reads (analogous to pasting one word in the middle of another) and most importantly reversed sequences from cDNAs threaded through the pores in 3′→5′ direction. If one were to limit attention to reads that conform to the ideal of a well-formed read, one would have to discard on average 45% of the counts in each library, thus further compressing the dynamic range and reducing the quantitative information that can be extracted from Nanopore libraries. To rescue the entire library for quantification, one must accommodate all these variations from the ideal read, which we did by developing a text-based segmentation algorithm that considered all possible deviations from the ideal read.

The first step in our segmentation algorithm is the identification of the location and orientation of the adapters that decorate the insert. The adapter identification step in the PALS-NS operates under similar principles to adapter trimming methods for short-read e.g. cutadapt, trimmomatic and long-read sequencing platforms such as Porechop, Pychopper and primer-chop, i.e. it is a gap alignment based method. The sound statistical properties of the blastn aligner we used control the false positive hits against the decorator sequences. Once an alignment has been found, our algorithm extends it to the entire length of the decorators (a form of semi-global alignment). In doing so, we thus fully account for sequencing and basecalling errors that may manifest as internal gaps in the alignment or as abbreviated alignments that don't cover the entire decorator length. A subtle point in this process, which sets our approach apart from others is our handling of the last four bases in 3′ end of 5′ adapter. This is where the RT switches templates and adds non-templated nucleotides. Previous work that examined the template switching junction has shown both sequence dependent bias and variable numbers of non-template nucleotides added. Therefore, neither the length, nor the composition of the junction can be taken as granted, and thus we opted to retain this feature as part of the insert sequence during text segmentation. This decision guards against the artificial shortening of short RNAs with multiple guanines in their 5′ end, which could have a detrimental impact on their identification during a database similarity search.

The second step in our algorithm, i.e., the reorientation of the insert is an area that has so far attracted limited attention. The only relevant works in this area are ONT's Pychopper, primer-chop and ReorientExpress. The latter is a neural network-based tool that was introduced on the premise that it can identify orientation with higher accuracy than Pychopper and primer-chop. We have not undertaken a direct comparison against these methods, because our text-based segmentation is well suited for the purpose of quantification; only does it rescue all reads (e.g., the current version primer-chop can't rescue fusion reads), but also appears to do so in a manner that does not compromise quantitation. Hence, the complexity of the dynamic programming algorithm used by pychopper to rescue reads, or the neural network-based method appear to be over-complicated for a task that is solvable by our approach.

The identification and elimination of the polyA tails via regular expression matching is a unique feature of our workflow and should be contrasted to the fixed length poly-A tail used in primer-chop or the fixed length cutadapt methods adopted in CATS/D-Plex and Smart-seq-total. These methods assume that all RNAs will have equal length poly-A, an assumption that is clearly not justified by the variation in the poly-A tails for the RNA species that are naturally poly-adenylated, the performance of the PAP enzyme in-vitro or by the non-uniform poly-A tail noted in our own data. On the other hand, our approach allows the poly-A tail to be of variable length, while accommodating a limited number of sequencing errors through the incorporation of non-A patterns in the expressions to be matched. We opine that the meticulous attention to the removal of the decorator and poly-A sequences underline the substantial enhanced mapping rate (over 50% and up to 70%) in the nanogram input libraries, vs. a figure less than 20% that was previously reported. One may wonder whether further enhancements in poly-A detection and removal could improve this mapping rate even further. In that regards, it should be noted that one can use probabilistic methods in the raw, electrical signal (“squiggle”) space to determine the poly-A length. While more sophisticated than our proposal, these approaches use specific features of the basecallers in the ONT software platforms and thus will likely have to be calibrated to future implementation of the basecalling suites. On the other hand, as our approach operates on the basecalled sequence, it is independent of the specific basecaller used. However improved approaches to identify and eliminate the poly-A tail, e.g. via different regular expression patterns or Hidden Markov Models are possible, and are currently the subject of investigation by our group.

PALS-NS extends the scope of long Nanopore reads to short non-coding and long coding and non-coding RNAs. Using synthetic mixes of short (microRNA) and long (ERCC) RNAs we demonstrated that PALS-NS can reliably detect both RNAs in proportion to their input amount. This proportionality is afforded by the non-selectivity of the PAP enzyme for the sequence at 3′ end of its substrates. These findings are not likely to be a chance event, because they were obtained in experiments that used sham poly-adenylation and sham short RNA input points to guard against chance variation. The merit of the polyadenylation step in extending the spectrum of Nanopore sequencing was shown in biological samples that were simultaneously sequenced on a short read platform and the unmodified SMART-seq like library preparation kit provided by ONT. PALS-NS detected the entire spectrum of RNAs present in these cellular sources while achieving a balanced ratio between coding and non-coding (e.g., lncRNA/microRNA) counts. An interesting and somewhat novel observation in both the synthetic and biological samples was that the length of the inserts mappable to long RNAs was not always in step with the expected size. Furthermore, the size attained was dependent on both the composition of the sample and the application of a PAP step. These findings were most clearly illustrated in the synthetic samples, and the most plausible explanation is premature template switching in the RT reaction. The mechanism for this premature switching is quite likely a direct competition between short heteroduplexes and partially extended long ones for the RT enzyme. There are several observations that argue in favor of this explanation: firstly this “shortening”, is not observed in the sham poly-adenylated or poly-adenylated samples devoid of microRNAs, except for very long RNAs. In the first samples the microRNAs are “invisible” to the RT reaction and in the second samples they are simply not present. In both cases, there are no heteroduplexes to complete for access to the enzyme. Secondly, the amount of shortening depends on the relative ratio of short and long RNAs, demonstrating the quantitative relation expected from a competitive reaction mechanism. Thirdly, this shortening is also observed for pure long RNAs libraries; in that case, intermediate length sequences play the role of short RNAs and limit the length of inserts derived from very long sequences. To our knowledge this is the first time that one reports this finding in RNA Nanopore sequencing, but the mechanism appears general and likely apply to all long-read protocols that utilize a RT step. While the premature termination of RT is certainly an undesirable feature for long read sequencing if the focus is to characterize the sequence of the transcripts, it does not impair the ability to quantitate RNAs if an alignment method is used to map the inserts against a reference database with statistical rigor. While this competition would seem to limit the case for using a long-read platform, one should note that the amount of shortening will be less in biological samples, than the extreme testing presented by our 2×2 and DS experiments. In fact, using the known composition of the ERCC we observed minimal shortening for RNAs smaller than 624 bases, while more than 50% of inserts deriving from ERCC RNAs between 624-1000 bp were of the expected length when the ERCC was spiked in biological samples.

When total RNA is used at the starting material for PALS-NS, the libraries will include numerous counts mapping to “undesirable” RNAs such as ribosomal RNAs and tRNAs; the known discrimination of PAP against 3′ stem loop structures (78-80) was not sufficient to prevent the tailing of molecules with such features despite the presence of multiple other substrates in the biological samples. Hence, if interest lies in the simultaneous profiling of coding and non-coding (lncRNA or microRNAs) the undesirable RNAs should be eliminated to preserve library depth. This can be achieved during sample preparation via RNAse H based treatment or via depletion by CRISPR-Cas9 after the cDNA library has been generated. On the other hand, the non-selective nature of poly-adenylation affords the opportunity to develop new sequencing protocols for the Nanopore platform e.g., by combining size selection and poly-A depletion to sequence non-coding RNAs with defined lengths. The ease by which tRNAs are adenylated by this enzyme, a property known since the 1970s, would allow for example the PALS-NS to be used as a novel technique for RNA quantitative sequencing, an area in dire need of flexible, non-tedious, high-resolution protocols. Other possible applications could include sequencing of predominantly non-coding epigenetically relevant RNAs, irrespective of length, after depletion of coding RNAs. The feasibility of such an application was clearly illustrated in the 2×2 experiments that utilized a large excess of microRNAs, to simulate a sample preparation in which the coding RNAs had been depleted prior to library preparation. Considering the emerging role of non-coding short RNAs other than microRNAs in the pathogenesis of disease, the PALS-NS protocol offers a unique opportunity to study such RNAs vis-à-vis lncRNAs and short non coding RNAs other than microRNAs. Such studies may allow a better mechanistic understanding(8) of a wide range of disorders and even Nanopore based biomarker assays.

PALS-NS shows a minimal amount of sequence and length dependent bias for either short or long RNA quantification: Our analyses probed the bias in PALS-NS and of the unmodified cDNA-PCR sequencing workflow for Nanopore devices. This bias may be conceptualized as a deviation of the observed counts of a given cDNA from those expected on the amount of the corresponding RNA present in the original sample. Such deviations may arise from the length of the RNA molecule (“length bias”) or poorly characterized sequence dependent factors (“sequence bias”). In an earlier publication, we introduced the term “bias factor” for this deviation and represented it as a random effect in over-dispersed Poisson (negative binomial) in the context of a ligation based, degenerate/randomized end 4N short RNA sequencing protocol. These protocols were the best performing methods in a multi-center evaluation of methods for quantitative microRNA sequencing and performed very well in single cell applications. Hence the observation that PALS-NS generates a similar magnitude of bias as one of the highly performing short RNA protocol is encouraging. However, these findings warrants replication and independent verification.

A unique finding of our work relates to the quantification of the length dependent bias of the PALS-NS protocol. This bias, which is a linear, deterministic, function of the logarithm of the length of the sequence does not appear to be present in the unmodified RNAseq protocol suggested by ONT, but it does manifest in PALS-NS as an over-representation of longer sequences relative to the shorter ones. We speculate that this bias materializes after poly-adenylation because of the capture of 5′ and mid-sequence fragments of the long RNAs. Lacking a poly-A tail, such fragments would not be represented in a protocol that does not include a poly-adenylation step but inflate the counts of long RNAs during PALS-NS after they acquire such a tail. Stated in other terms, we hypothesize that this is a form of “fragmentation”, length-dependent bias similar to that seen in short RNA sequencing platforms. This hypothesis can be resolved by a meticulous alignment analysis of the captured fragments under conditions of sham and actual poly-adenylation. Space considerations preclude us from carrying out such an analysis in this report. Regardless of the mechanisms that lead to such bias, it should be noted that the magnitude is rather small and at least an order of magnitude less than the length dependent bias that is seen with much more expensive sequencing platforms, e.g. the NextSeq and Novaseq.

PALS-NS is a suitable approach for epigenetic research: Our main impetus in developing PALS-NS is to allow simultaneous analysis of non-coding and coding RNAs from a single library preparation for epigenetic research. The molecular biology techniques required to profile short and long RNAs are rather different, thus simultaneous profiling in either bulk or single cell samples requires duplicate workflows, and even different measurement techniques. These may include for example combining RTPCR with microarrays or running separate libraries in the case of sequencing. To our knowledge, the only two publications exploring simultaneous profiling of non-coding and coding RNAs, CATS and Smart-seq-total target the Illumina sequencing platforms. CATS is one of the first papers to explore a PAP protocol and despite the use of older generation RT with substantial RNAseH activity is worth pointing that it achieved a rather large percentage of mappable reads (more than 65), but no information was provided about the microRNAs vs. coding RNAs in the resultant libraries. Like our paper, Smart-seq-total profiled the entire complement of RNA and provides independent verification of the validity of our approach. However, at closer look there are some notable differences between the results reported by the Smart-seq-total investigators and the findings reported here-in that merit consideration. In particular, the ratio of coding/lncRNA/microRNA/snoRNAs/snRNAs as % of the rRNA depleted libraries was reported as 50:1:0.4:1:1, whereas the corresponding ratio was 29:11:2.1:1:0.2. While the source of the RNA (bulk in our case, single cells in Smart-seq-total), and sequencing at a different depth with a short read platform may underline these differences, a careful examination of the quantitative aspects of the Smart-seq-total report and this report suggests that such differences may be protocol dependent. Even though the library depths differed between the Smart-seq-total (2.5M reads) and our protocol (4.3M and 8.5M), many reads in our libraries (˜40%) mapped to ribosomal RNAs, so that the non-ribosomal library depth we obtained was rather comparable to the Smart-seq-total paper. In support of the latter assertion, examination of the ERCC control counts-molarity curve shows that the dynamic range compression occurred at roughly similar points, i.e. at 3.5 log 10 from the least expressed ERCC RNA. Since the number of all RNA biotypes was reduced in the single cell RNA libraries, relative to the bulk, but the library depth was comparable, one would have expected the ratio of read types to be rather similar between Smart-seq-total and PALS-NS. The observation that it is not, suggests that the Smart-seq-total protocol may not be as efficient as PALS-NS in capturing both short and long non-coding RNAs because of biochemistry (tagmentation/pooling, capping reaction), bead clean-up (use of 1.0× volume of beads vs. 1.8×), CRISPR-Cas9 library depletion or the sequencing platform (Novaseq vs Nanopore). While we cannot resolve these differences without further exploration of our protocol in single cell applications, the more balanced representation between coding and non-coding RNAs suggest that the Nanopore based PALS-NS may be more suitable for resolving non-coding RNAs than the Illumina based Smart-seq-total for bulk RNA sequencing.

PALS-NS demonstrates a very high dynamic range yet requires further optimization for low input samples. A key observation is the extremely high dynamic range of the PALS-NS, which can generate libraries in which the representation of molecules scales linear with abundance over eight orders of magnitude, i.e., much higher than the dynamic range of most, except the very high-end sequencing flow cells. As the relative cost of Nanopore sequencing and the capabilities of the flow cells continue to improve, PALS-NS is well positioned to quantitate RNAs without the library depth limitations of current flow cells. Nevertheless, certain challenges remain to be addressed for low input (3-30 pg) samples, that while easily handled by the protocol, tend to generate a high number of adapter dimers and non-mappable reads. Dimers can be reduced at the magnetic bead clean up stage e.g., by decreasing the ratio of beads to sample volume from 1.8× closer to 1.0× at the expense of losing a variable amount of the short RNA derived inserts. On the other hand, the high percentage of non-mappable reads may require optimization of the PAP and RT steps as these reads likely originate at the interface of these reactions. Previous work has shown that while the mapping rate of Maxima Minus H derived libraries will be in the 85-90% range for ng input (also observed in our work), the mapping rate will decline to ˜50% in the pigogram range. Mapping of low input PALS-NS libraries such as the 1:100 and 1:1000 diluted synthetic mixes was much lower, suggesting that the high number of non-mappable reads may originate at this stage. It should be noted that similar observations, i.e. high dimers and a large proportion of nonmappable reads were made in an evaluation of the CATS protocol for the ultimate low input sample, i.e. single cell microRNA sequencing. Hence, extending the scope of PALS-NS to low input or even single cell RNA sequencing applications will require further kinetic or input optimization of the PAP reaction and possibly exploration of alternative reverse transcriptase enzymes.

### CONCLUSIONS

1. PALS-NS is capable of simultaneously profiling short and long RNAs from a single tube reaction through a simple PAP modification of existing SMART-seq protocols and associated bioinformatics workflow using Nanopore sequences.

2. PALS-NS extends the dynamic range of reads detection to non-coding RNAs with limited length and sequence-dependent bias.

3. Bias for short RNAs is comparable to the gold standard Illumina protocol (4N) developed by NIH's exRNA consortium.

4. The entire complement of RNAs in biological samples is profiled by PALS-NS in bulk RNAseq applications.

5. Future adaptations of the protocol may extend its scope for (ultra-) low input samples and single cell RNA sequencing.

Some further aspects are defined in the following clauses:

Clause 1: A method of substantially simultaneously detecting coding and non-coding linear ribonucleic acids (RNAs) in a sample. The method comprises attaching a polymeric nucleic acid tail to a plurality of the non-coding linear RNAs in the sample, wherein the sample comprises the coding and non-coding linear RNAs irrespective of lengths of the RNAs, to produce a population of RNA molecules that each comprise polymeric nucleic acid tails; and obtaining sequence information from the population of RNA molecules that each comprise polymeric nucleic acid tails and/or from derivative nucleic acid molecules thereof irrespective of lengths of the RNA molecules or the derivative nucleic acid molecules thereof using a long read sequencing technique, thereby substantially simultaneously detecting the coding and non-coding linear RNAs in the sample.

Clause 2: A method of processing sequencing reads. The method comprises attaching a polymeric nucleic acid tail to a plurality of the non-coding RNAs in a sample, wherein the sample comprises coding and non-coding ribonucleic acids (RNAs), to produce a population of RNA molecules that each comprise polymeric nucleic acid tails, and obtaining sequencing reads from the population of RNA molecules that each comprise polymeric nucleic acid tails and/or from derivative nucleic acid molecules thereof. The method also comprises differentiating decorator sequence information from insert sequence information in the plurality of sequencing reads, and determining orientations of subsequences in the plurality of sequencing reads corresponding to the coding and non-coding RNAs using the decorator and/or insert sequence information, thereby the processing sequencing reads.

Clause 3: A method of mapping sequence information to a genomic transcriptome using a computer. The method comprises receiving, by the computer, sequencing reads from a population of ribonucleic acid (RNA) molecules that each comprise polymeric nucleic acid tails and/or from derivative nucleic acid molecules thereof, wherein the RNA molecules comprise coding and non-coding RNAs, differentiating, by the computer, decorator sequence information from insert sequence information in the plurality of sequencing reads, and determining, by the computer, orientations of coding RNA sequence information and non-coding RNA sequence information from the insert sequence information. The method also comprises removing or disregarding, by the computer, decorator sequence information from the insert sequence information to produce processed insert sequence information, and mapping, by the computer, the processed insert sequence information to a selected genomic transcriptome, thereby mapping the sequence information to the genomic transcriptome.

Clause 4: A method of detecting non-coding linear ribonucleic acids (RNAs) in a sample. The method comprises attaching a polymeric nucleic acid tail to a plurality of the non-coding linear RNAs in the sample, wherein the sample comprises the coding and non-coding linear RNAs irrespective of lengths of the RNAs, to produce a population of RNA molecules that each comprise polymeric nucleic acid tails, and obtaining sequence information from the population of RNA molecules that each comprise polymeric nucleic acid tails and/or from derivative nucleic acid molecules thereof irrespective of lengths of the RNA molecules or the derivative nucleic acid molecules thereof using a sequencing technique, thereby detecting the non-coding linear RNAs in the sample.

Clause 5: A method of substantially simultaneously detecting coding and non-coding linear ribonucleic acids (RNAs) in a sample. The method comprises processing the coding and non-coding linear RNAs irrespective of lengths of the RNAs in the sample in a single reaction container to produce a population of processed RNA molecules, and obtaining sequence information from the population of processed RNA molecules using a sequencing technique, thereby substantially simultaneously detecting the coding and non-coding linear RNAs in the sample.

Clause 6: The method of any one of the preceding Clauses 1-5, wherein the polymeric nucleic acid tail comprises a homopolymeric nucleic acid tail.

Clause 7: The method of any one of the preceding Clauses 1-6, wherein the homopolymeric nucleic acid tail comprises a poly-A, poly-C, poly-U or poly-G nucleic acid tail.

Clause 8: The method of any one of the preceding Clauses 1-7, wherein the decorator sequence information corresponds to nucleic acid sequences attached to the RNA molecules after obtaining the sample.

Clause 9: The method of any one of the preceding Clauses 1-8, wherein the decorator sequence information corresponds to primer nucleic acid sequences, polymeric nucleic acid tail sequences, adapter nucleic acid sequences, or barcode nucleic acid sequences.

Clause 10: The method of any one of the preceding Clauses 1-9, comprising performing the attaching step of the polymeric nucleic acid tail and one or more polymerase chain reaction (PCR) steps in a single reaction container.

Clause 11: The method of any one of the preceding Clauses 1-10, further comprising size selecting the coding and non-coding RNAs in the sample to comprise longer and shorter RNA molecules of selected nucleotide lengths prior to obtaining the sequence information.

Clause 12: The method of any one of the preceding Clauses 1-11, further comprising separating the coding and non-coding RNAs from one or more other components of the sample prior to attaching the polymeric nucleic acid tail to the plurality of the non-coding RNAs in the sample.

Clause 13: The method of any one of the preceding Clauses 1-12, wherein the other components comprise ribosomal RNAs (rRNAs), transfer RNAs (tRNAs), microRNAs (miRNAs), piwi RNAs (piRNAs), and any linear coding and non-coding RNAs present in the sample.

Clause 14: The method of any one of the preceding Clauses 1-13, further comprising determining relative amounts of the coding and non-coding RNAs in the sample.

Clause 15: The method of any one of the preceding Clauses 1-14, further comprising attaching one or more adapters to the RNA molecules that each comprise polymeric nucleic acid tails and/or to the derivative nucleic acid molecules thereof prior to obtaining the sequence information.

Clause 16: The method of any one of the preceding Clauses 1-15, wherein the coding RNAs in the sample comprise poly-A nucleic acid tail sub-sequences prior to attaching the polymeric nucleic acid tail to the plurality of the non-coding RNAs in the sample.

Clause 17: The method of any one of the preceding Clauses 1-16, wherein the coding RNAs comprise messenger RNAs (mRNAs).

Clause 18: The method of any one of the preceding Clauses 1-17, wherein the coding RNAs are long RNAs that comprise a mean length that is greater than about 50, about 100, about 150, about 200, about 250, about 300, about 350, or more nucleotides.

Clause 19: The method of any one of the preceding Clauses 1-18, wherein the non-coding RNAs comprise linear RNA molecules.

Clause 20: The method of any one of the preceding Clauses 1-19, wherein the non-coding RNAs comprise microRNAs (miRNAs).

Clause 21: The method of any one of the preceding Clauses 1-20, wherein the non-coding RNAs are short RNAs that comprise a mean length that is less than about 50, about 40, about 30, about 20, or fewer nucleotides.

Clause 22: The method of any one of the preceding Clauses 1-21, wherein the derivative nucleic acid molecules thereof comprise complementary deoxyribonucleic acid (cDNA) molecules.

Clause 23: The method of any one of the preceding Clauses 1-22, wherein the sample is obtained from a subject.

Clause 24: The method of any one of the preceding Clauses 1-23, wherein the obtaining step comprises using at least one PCR-cDNA sequencing technique.

Clause 25: The method of any one of the preceding Clauses 1-24, wherein the obtaining step comprises using at least one next generation sequencing technique.

Clause 26: The method of any one of the preceding Clauses 1-25, wherein the next generation sequencing technique comprises at least one nanopore sequencing technique.

Clause 27: The method of any one of the preceding Clauses 1-26, wherein the next generation sequencing technique comprises at least one single molecule sequencing technique.

Clause 28: The method of any one of the preceding Clauses 1-27, wherein the sequence information comprises a plurality of sequencing reads and wherein the method further comprises determining orientations of coding RNA sequence information and non-coding RNA sequence information from the plurality of sequencing reads.

Clause 29: The method of any one of the preceding Clauses 1-28, wherein the determining step comprises identifying sequencing reads corresponding to the coding and non-coding RNAs and identifying sequencing reads corresponding to complements or reverse complements of the coding and non-coding RNAs.

Clause 30: The method of any one of the preceding Clauses 1-29, further comprising mapping at least a portion of the sequence information to a genomic transcriptome.

Clause 31: The method of any one of the preceding Clauses 1-30, further comprising differentiating decorator sequence information from insert sequence information using the plurality of sequencing reads.

Clause 32: The method of any one of the preceding Clauses 1-31, wherein the decorator sequence information corresponds to poly-A, poly-C, poly-U or poly-G nucleic acid tails of the coding and non-coding RNAs and/or to one or more adapters attached to the coding and non-coding RNAs using a non-templated nucleic acid polymerase.

Clause 33: The method of any one of the preceding Clauses 1-32, wherein determining the orientations of coding RNA sequence information and non-coding RNA sequence information and differentiating the decorator sequence information from the insert sequence information comprises combining a sequence alignment technique with an expression matching technique.

Clause 34: The method of any one of the preceding Clauses 1-33, wherein the differentiating step comprises using at least one text view technique.

Clause 35: The method of any one of the preceding Clauses 1-34, wherein the insert sequence information comprises the coding RNA sequence information and non-coding RNA sequence information.

Clause 36: The method of any one of the preceding Clauses 1-35, further comprising determining orientations of subsequences in the plurality of sequencing reads corresponding to the coding and non-coding RNAs using at least the insert sequence information, thereby the processing sequencing reads.

Clause 37: The method of any one of the preceding Clauses 1-36, further comprising re-orienting the subsequences in the plurality of sequencing reads corresponding to the coding and non-coding RNAs that are determined to be in a 3′ to 5′ orientation to a 5′ to 3′ orientation.

Clause 38: The method of any one of the preceding Clauses 1-37, wherein the determining step comprises identifying whether the insert information is in a sense direction or in an antisense direction.

Clause 39: The method of any one of the preceding Clauses 1-38, wherein the sequence information comprises a plurality of sequencing reads and wherein the method further comprising determining whether a given sequencing read is a well-formed sequencing read, a partial sequencing read, a naked sequencing read, or a fusion sequencing read.

Clause 40: A system, comprising at least one controller that comprises, or is capable of accessing, computer readable media comprising non-transitory computer-executable instructions which, when executed by at least one electronic processor, perform at least: receiving sequencing reads from a population of ribonucleic acid (RNA) molecules that each comprise polymeric nucleic acid tails and/or from derivative nucleic acid molecules thereof, wherein the RNA molecules comprise coding and non-coding RNAs, differentiating decorator sequence information from insert sequence information in the plurality of sequencing reads, and determining orientations of subsequences in the plurality of sequencing reads corresponding to the coding and non-coding RNAs using at least the insert sequence information.

Clause 41: A system, comprising at least one controller that comprises, or is capable of accessing, computer readable media comprising non-transitory computer-executable instructions which, when executed by at least one electronic processor, perform at least: receiving sequencing reads from a population of ribonucleic acid (RNA) molecules that each comprise polymeric nucleic acid tails and/or from derivative nucleic acid molecules thereof, wherein the RNA molecules comprise coding and non-coding RNAs, differentiating decorator sequence information from insert sequence information in the plurality of sequencing reads, determining orientations of coding RNA sequence information and non-coding RNA sequence information from the insert sequence information, removing or disregarding decorator sequence information from the insert sequence information to produce processed insert sequence information, and mapping the processed insert sequence information to a selected genomic transcriptome, thereby mapping the sequence information to the genomic transcriptome.

Clause 42: A computer readable media comprising non-transitory computer-executable instructions which, when executed by at least one electronic processor, perform at least: receiving sequencing reads from a population of ribonucleic acid (RNA) molecules that each comprise polymeric nucleic acid tails and/or from derivative nucleic acid molecules thereof, wherein the RNA molecules comprise coding and non-coding RNAs, differentiating decorator sequence information from insert sequence information in the plurality of sequencing reads, and determining orientations of subsequences in the plurality of sequencing reads corresponding to the coding and non-coding RNAs using at least the insert sequence information.

Clause 43: A computer readable media comprising non-transitory computer-executable instructions which, when executed by at least one electronic processor, perform at least: receiving sequencing reads from a population of ribonucleic acid (RNA) molecules that each comprise polymeric nucleic acid tails and/or from derivative nucleic acid molecules thereof, wherein the RNA molecules comprise coding and non-coding RNAs, differentiating decorator sequence information from insert sequence information in the plurality of sequencing reads, determining orientations of coding RNA sequence information and non-coding RNA sequence information from the insert sequence information, removing or disregarding decorator sequence information from the insert sequence information to produce processed insert sequence information, and mapping the processed insert sequence information to a selected genomic transcriptome, thereby mapping the sequence information to the genomic transcriptome.

While the foregoing disclosure has been described in some detail by way of illustration and example for purposes of clarity and understanding, it will be clear to one of ordinary skill in the art from a reading of this disclosure that various changes in form and detail can be made without departing from the true scope of the disclosure and may be practiced within the scope of the appended claims. For example, all the methods, systems, computer readable media, and/or component features, steps, elements, or other aspects thereof can be used in various combinations.

All patents, patent applications, websites, other publications or documents, accession numbers and the like cited herein are incorporated by reference in their entirety for all purposes to the same extent as if each individual item were specifically and individually indicated to be so incorporated by reference. If different versions of a sequence are associated with an accession number at different times, the version associated with the accession number at the effective filing date of this application is meant. The effective filing date means the earlier of the actual filing date or filing date of a priority application referring to the accession number, if applicable. Likewise if different versions of a publication, website or the like are published at different times, the version most recently published at the effective filing date of the application is meant, unless otherwise indicated.

1. A method of substantially simultaneously detecting coding and non-coding linear ribonucleic acids (RNAs) in a sample, the method comprising:
attaching a polymeric nucleic acid tail to a plurality of the non-coding linear RNAs in the sample, wherein the sample comprises the coding and non-coding linear RNAs irrespective of lengths of the RNAs, to produce a population of RNA molecules that each comprise polymeric nucleic acid tails; and,
obtaining sequence information from the population of RNA molecules that each comprise polymeric nucleic acid tails and/or from derivative nucleic acid molecules thereof irrespective of lengths of the RNA molecules or the derivative nucleic acid molecules thereof using a long read sequencing technique, thereby substantially simultaneously detecting the coding and non-coding linear RNAs in the sample.

2. The method of claim 1, wherein the polymeric nucleic acid tail comprises a homopolymeric nucleic acid tail.

3. The method of claim 2, wherein the homopolymeric nucleic acid tail comprises a poly-A, poly-C, poly-U or poly-G nucleic acid tail.

4. The method of claim 1, comprising performing the attaching step of the polymeric nucleic acid tail and one or more polymerase chain reaction (PCR) steps in a single reaction container.

5. The method of claim 1, further comprising size selecting the coding and non-coding RNAs in the sample to comprise longer and shorter RNA molecules of selected nucleotide lengths prior to obtaining the sequence information.

6. The method of claim 1, further comprising separating the coding and non-coding RNAs from one or more other components of the sample prior to attaching the polymeric nucleic acid tail to the plurality of the non-coding RNAs in the sample.

7. The method of claim 6, wherein the other components comprise ribosomal RNAs (rRNAs), transfer RNAs (tRNAs), microRNAs (miRNAs), piwi RNAs (piRNAs), and any linear coding and non-coding RNAs present in the sample.

8. The method of claim 1, further comprising determining relative amounts of the coding and non-coding RNAs in the sample.

9. The method of claim 1, further comprising attaching one or more adapters to the RNA molecules that each comprise polymeric nucleic acid tails and/or to the derivative nucleic acid molecules thereof prior to obtaining the sequence information.

10. The method of claim 1, wherein:
the coding RNAs in the sample comprise poly-A nucleic acid tail sub-sequences prior to attaching the polymeric nucleic acid tail to the plurality of the non-coding RNAs in the sample;
the coding RNAs comprise messenger RNAs (mRNAs); or,
the coding RNAs are long RNAs that comprise a mean length that is greater than about 50, about 100, about 150, about 200, about 250, about 300, about 350, or more nucleotides.

11. (canceled)

12. (canceled)

13. The method of claim 1, wherein:
the non-coding RNAs comprise microRNAs (miRNAs); or,
the non-coding RNAs are short RNAs that comprise a mean length that is less than about 50, about 40, about 30, about 20, or fewer nucleotides.

14. (canceled)

15. The method of claim 1, wherein the derivative nucleic acid molecules thereof comprise complementary deoxyribonucleic acid (cDNA) molecules.

16. The method of claim 1, wherein the sample is obtained from a subject.

17. (canceled)

18. The method of claim 1, wherein the obtaining step comprises using at least one PCR-cDNA sequencing technique or using at least one next generation sequencing technique.

19. The method of claim 18, wherein the next generation sequencing technique comprises at least one nanopore sequencing technique or at least one single molecule sequencing technique.

20. (canceled)

21. The method of claim 1, wherein the sequence information comprises a plurality of sequencing reads and wherein the method further comprises determining orientations of coding RNA sequence information and non-coding RNA sequence information from the plurality of sequencing reads.

22. The method of claim 21, wherein the determining step comprises identifying sequencing reads corresponding to the coding and non-coding RNAs and identifying sequencing reads corresponding to complements or reverse complements of the coding and non-coding RNAs.

23. The method of claim 22, further comprising mapping at least a portion of the sequence information to a genomic transcriptome.

24.-59. (canceled)

60. A system, comprising at least one controller that comprises, or is capable of accessing, computer readable media comprising non-transitory computer-executable instructions which, when executed by at least one electronic processor, perform at least:
receiving sequencing reads from a population of ribonucleic acid (RNA) molecules that each comprise polymeric nucleic acid tails and/or from derivative nucleic acid molecules thereof, wherein the RNA molecules comprise coding and non-coding RNAs;
differentiating decorator sequence information from insert sequence information in the plurality of sequencing reads; and,
determining orientations of subsequences in the plurality of sequencing reads corresponding to the coding and non-coding RNAs using at least the insert sequence information.

61. (canceled)

62. A computer readable media comprising non-transitory computer-executable instructions which, when executed by at least one electronic processor, perform at least:
receiving sequencing reads from a population of ribonucleic acid (RNA) molecules that each comprise polymeric nucleic acid tails and/or from derivative nucleic acid molecules thereof, wherein the RNA molecules comprise coding and non-coding RNAs;
differentiating decorator sequence information from insert sequence information in the plurality of sequencing reads; and,
determining orientations of subsequences in the plurality of sequencing reads corresponding to the coding and non-coding RNAs using at least the insert sequence information.

63. (canceled)
